(19) United States
(12) Patent Application Publication
(10) Pub. No.: US 2022/0195061 A1
(43) Pub. Date: Jun. 23, 2022

(54) ANTI-CD40 ANTIBODIES FOR USE IN TREATMENT OF TIDM AND INSULITIS

(71) Applicant: NOVARTIS AG, Basel (CH)

(72) Inventors: Pascal ESPIE, Saint Louis (FR); Allison GOLDFINE, Wayland, MA (US); Rainer MUSSMANN, Basel (CH); James RUSH, Thalwil (CH)

(21) Appl. No.: 17/609,261

(22) PCT Filed: May 6, 2020
(86) PCT No.: PCT/IB2020/054271

§ 371 Date: Nov. 5, 2021

Related U.S. Application Data
(60) Provisional application No. 62/844,960, filed on May 8, 2019.

(51) Int. Cl. C07K 16/28; A61P 3/10

(52) U.S. Cl. CPC C07K 16/2878; A61K 2039/505; A61P 3/10

(57) ABSTRACT

The disclosure relates to methods, treatment regimens, uses, kits and therapies for treating T1DM, by employing anti-CD40 antibodies.

### TECHNICAL FIELD

The disclosure relates to methods, treatment regimens, uses, kits and therapies for treating T1DM or insulitis by employing anti-CD40 antibodies, such as CFZ533.

### BACKGROUND OF THE DISCLOSURE

Insulin replacement is life-saving and treats the symptoms of type 1 diabetes mellitus (T1DM) but does not alter disease progression. Around 132,600 children and adolescents develop T1DM each year worldwide (Cho et al 2018) and prevalence is rising, with an estimated 3% increase per year. The number of children with T1DM in Europe is projected to triple between 2010 and 2050 (Imperatore et al 2012). New onset T1DM can occur at any age but peak incidence occurs between the ages of 5 and 15 years. Life expectancy is reduced by 12 years on average compared to the general population (Secrest et al 2010). Long-term micro and macrovascular complications continue to be burden for T1DM patients, their families, and for society (Secrest et al 2010).

T1DM disease onset and progression over the first year are markedly more aggressive in pediatric versus adult patients. Pediatric patients with new onset T1DM present with lower residual β-cell function at disease onset and a more rapid decline in remaining β-cell function over the first year when compared to adults (Greenbaum et al 2012). Pediatric patients with new onset T1DM appear to be more responsive to immune modulatory interventions such as rituximab and teplizumab in randomized control trials (Woittiez and Roep 2015). Yet, despite technological and clinical advances in the standard of care of T1DM, the majority of patients and particularly pediatric patients are not meeting therapeutic (e.g. glycemic) targets (Miller et al 2015). Therapies preserving residual β-cell function have potential to improve glycemic control and avoid serious short and long complications associated with T1DM, but no satisfactory immune modulatory intervention based treatment of T1DM, which alters disease progression, in particular in pediatric patients, are available. Hence, there is a need for the development of an efficient treatment of T1DM, in particular for the treatment of pediatric patients.

CFZ533 is a human monoclonal antibody directed against human CD40. It belongs to the IgG1 isotype subclass with and comprises an Fc-silencing mutation (N297A) which abolishes FcγR binding and associated effector functions like ADCC and CDC. CFZ533 is disclosed in U.S. Pat. Nos. 8,828,396 and 9,221,913.

### SUMMARY OF THE DISCLOSURE

It is envisaged by the inventors that human anti-CD40 monoclonal antibodies with silenced ADCC activity are suitable for the treatment of T1DM and insulitis. Particularly, the antibody CFZ533 is believed to be particularly useful in a new modality for the treatment of T1DM and insulitis.

According to a first aspect of the disclosure an anti-CD40 antibody with silenced ADCC activity for use in the treatment of T1DM is provided, comprising administering a therapeutically effective amount of said antibody to a patient in need thereof, wherein said antibody is administered through a loading dosing followed by a maintenance dosing and the route of administration is subcutaneous or intravenous, or a combination of subcutaneous or intravenous.

In another embodiment, the antibody for use according to the first aspect of the disclosure is provided using a loading dosing administered via intravenous injection and using a maintenance dosing administered via subcutaneous injections.

In one embodiment, the antibody for use according to the first aspect of the disclosure is provided using a loading dosing administered via intravenous injection as a first dose and using a maintenance dosing administered via subcutaneous injections as a second dose being different from the first dose.

In one embodiment of the disclosure, the antibody for use according to the first aspect of the disclosure is administered using a loading dose of about 3 mg to about 60 mg antibody per kilogram of the patient.

In one embodiment of the disclosure, the antibody for use according to the first aspect of the disclosure is administered using a loading dose of about 10 mg to about 30 mg antibody per kilogram of the patient.

In another embodiment, the antibody for use according to the first aspect of the disclosure is administered to a pediatric patient.

In an additional embodiment, the antibody for use according to any of the above described embodiments of the first aspect of the disclosure is administered using a loading dose of 30 mg/kg administered intravenously at day 1 and using a maintenance dose that is administered subcutaneously as a fixed dose between 100 mg to 350 mg once weekly starting at day 8.

In an additional embodiment, the antibody for use according to any of the above-described embodiments of the first aspect of the disclosure is administered using a maintenance dose that is subcutaneously administered as a fixed dose by body weight once weekly starting at day 8 at a dose of:

a. 135 mg for patients of body weight category I, having a bodyweight between 20 kg to 30 kg;
b. 195 mg for patients of body weight category II, having a bodyweight between 30 kg to 50 kg; and
c. 300 mg for patients of body weight category III, having a bodyweight above 50 kg.

In one embodiment, the maintenance dose of the antibody for use according to any of the above-described embodiments of the first aspect of the disclosure is administered to patients of:

a) body weight category I in form of a single injection of 0.9 ml; and
b) body weight category II in form of a single injection of 1.3 ml; or
c) body weight category III in form of a single injection of 2 ml or 2 injections of 1 ml.

In an additional embodiment, the antibody for use according to any of the above-described embodiments of the first aspect of the disclosure is used in a treatment that lasts up to 52 weeks after day 1.

In an additional embodiment, the antibody for use according to any of the above-described embodiments of the first aspect of the disclosure is administered to patients of an age ranging between 6 and 21 years.

In one embodiment, the antibody for use according to any of the above-described embodiments of the first aspect of the disclosure is selected from the group consisting of:

a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

In one additional embodiment, the antibody for use according to any of the above described embodiments of the first aspect of the disclosure comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In an additional embodiment, the antibody for use according to any of the above-described embodiments of the first aspect of the disclosure is CFZ533.

In another embodiment, the antibody for use according to any of the above-described embodiments of the first aspect of the disclosure is administered to the patients within the first 100 days after diagnosis of T1DM in said patient. In another embodiment, the antibody for use according to any of the above-described embodiments of the first aspect of the disclosure is administered to the patients within the first 100 days after diagnosis of T1DM in said patient, but not before two weeks (14 days) after the diagnosis.

A second aspect of the disclosure relates to a pharmaceutical composition comprising a therapeutically effective amount of the antibody for use according to any of the above-described embodiments of the first aspect of the disclosure and one or more pharmaceutically acceptable carriers.

In a third aspect of the disclosure a method of treating T1DM in a human subject is provided, comprising administering a therapeutically effective dose of an anti-CD40 antibody with silenced ADCC activity to said subject, wherein said antibody is administered through a loading dosing followed by a maintenance dosing and the route of administration is subcutaneous or intravenous, or a combination of subcutaneous or intravenous.

In an additional embodiment, the method according to the third aspect of the disclosure comprises a loading dosing of a first dose of the anti-CD40 antibody that is administered via intravenous injection and a maintenance dosing of the anti-CD40 antibody of a second dose that is administered via subcutaneous injections, wherein the second dose is different from the first dose.

In one embodiment, the method according to any of the above described embodiments of the third aspect of the disclosure comprises a loading dose of the anti-CD40 antibody of about 3 mg to about 30 mg antibody per kilogram of the patient.

In another embodiment, the patient treated according to the method of any of the above-described embodiments of the third aspect of the disclosure is a pediatric patient.

In an additional embodiment, the method according to any of the above-described embodiments of the third aspect of the disclosure comprises administration of a loading dose of 30 mg/kg of the anti-CD40 antibody administered intravenously at day 1 and administration of a maintenance dose of the anti-CD40 antibody administered subcutaneously as a fixed dose between 100 mg-350 mg once weekly starting at day 8.

In another embodiment, the method according to any of the above-described embodiments of the third aspect of the disclosure comprises subcutaneous administration of a maintenance dose of the anti-CD40 antibody as a fixed dose by body weight once weekly starting at day 8 at a dose of:

a. 135 mg for patients of body weight category I, having a bodyweight between 20 to 30 kg;
b. 195 mg for patients of body weight category II, having a bodyweight between 30 to 50 kg; and
c. 300 mg for patients of body weight category III, having a bodyweight above 50 kg.

In an additional embodiment, the method according to any of the above described embodiments of the third aspect of the disclosure comprises administration of the anti-CD40 antibody to the patients of the

a) body weight category I in form of a single injection of 0.9 ml; and
b) body weight category II in form of a single injection of 1.3 ml; or
c) body weight category III in form of a single injection of 2 ml or 2 injections of 1 ml.

In another embodiment, the method according to any of the above-described embodiments of the third aspect of the disclosure comprises a treatment of the patients with the anti-CD40 antibody for up to 52 weeks after day 1.

In one embodiment, the age range of the patients treated using the method according to any of the above-described embodiments of the third aspect is between 6 and 21 years.

In another embodiment, the anti-CD40 antibody used in the method according to any of the above-described embodiments of the third aspect of the disclosure is selected from the group consisting of:

a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

In an additional embodiment, the anti-CD40 antibody used in the method according to any of the above described embodiments of the third aspect of the disclosure comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In one embodiment, the anti-CD40 antibody used in the method according to any of the above described embodiments of the third aspect of the disclosure is CFZ533.

In another embodiment, the antibody used in the method according to any of the above-described embodiments of the third aspect of the disclosure is administered to the patients within the first 100 days after diagnosis of T1DM in said patient.

In another embodiment, the antibody for use according to any of the above-described embodiments of the third aspect of the disclosure is administered to the patients within the first 100 days after diagnosis of T1DM in said patient, but not before two weeks (14 days) after the diagnosis.

A fourth aspect of the disclosure relates to the use of a liquid pharmaceutical composition comprising an anti-CD40 antibody with silenced ADCC activity, a buffer, a stabilizer and a solubilizer, for the manufacture of a medicament for the treatment of T1DM, wherein the anti-CD40 antibody is selected from the group consisting of:

i. an anti-CD40 antibody comprising an immunoglobulin VH domain comprising the amino acid sequence of SEQ ID NO: 7 and an immunoglobulin VL domain comprising the amino acid sequence of SEQ ID NO: 8;
ii. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
iii. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
iv. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14;
v. an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12, and vi. CFZ533.

In an additional embodiment the fourth aspect of the disclosure relates to the use of a liquid pharmaceutical composition comprising an anti-CD40 antibody, a buffer, a stabilizer and a solubilizer, for the manufacture of a medicament for the treatment of T1DM, wherein the anti-CD40 antibody:

a. is to be intravenously administered with a first loading dosing; and
b. thereafter, with a second maintenance dosing regimen administered subcutaneously, wherein said anti-CD40 antibody is selected from the group consisting of:
i. an anti-CD40 antibody comprising an immunoglobulin VH domain comprising the amino acid sequence of SEQ ID NO: 7 and an immunoglobulin VL domain comprising the amino acid sequence of SEQ ID NO: 8;
ii. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
iii. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
iv. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14;
v. an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12, and vi. CFZ533.

In another embodiment, the antibody used in the manufacture of a medicament according to any of the above-described embodiments of the fourth aspect of the disclosure is administered in a first loading dose to the patients in within the first 100 days after diagnosis of T1DM in said patient.

In another embodiment, the antibody used in the manufacture of a medicament according to the above-described embodiments of the fourth aspect of the disclosure is administered to the patients within the first 100 days after diagnosis of T1DM in said patient, but not before two weeks (14 days) after the diagnosis.

In a fifth aspect the disclosure relates to an anti-CD40 antibody with silenced ADCC activity for use in the treatment of insulitis, comprising administering a therapeutically effective amount of said antibody to a patient in need thereof, wherein said antibody is administered through a loading dosing followed by a maintenance dosing and the route of administration is subcutaneous or intravenous, or a combination of subcutaneous or intravenous.

In one embodiment, the antibody for use according to the fifth aspect of the disclosure, is provided using a loading dosing administered via intravenous injection as a first dose and using a maintenance dosing administered via subcutaneous injections as a second dose being different from the first dose.

In one embodiment of the disclosure, the antibody for use according to the fifth aspect of the disclosure is administered using a loading dose of about 3 mg to about 60 mg antibody per kilogram of the patient.

In one embodiment of the disclosure, the antibody for use according to the fifth aspect of the disclosure is administered using a loading dose of about 10 mg to about 30 mg antibody per kilogram of the patient.

In another embodiment, the antibody for use according to the fifth aspect of the disclosure is administered to a pediatric patient.

In an additional embodiment, the antibody for use according to any of the above described embodiments of the fifth aspect of the disclosure is administered using a loading dose of 30 mg/kg administered intravenously at day 1 and using a maintenance dose that is administered subcutaneously as a fixed dose between 100 mg to 350 mg once weekly starting at day 8.

In an additional embodiment, the antibody for use according to any of the above-described embodiments of the fifth aspect of the disclosure is administered using a maintenance dose that is subcutaneously administered as a fixed dose by body weight once weekly starting at day 8 at a dose of:

a. 135 mg for patients of body weight category I, having a bodyweight between 20 kg to 30 kg;
b. 195 mg for patients of body weight category II, having a bodyweight between 30 kg to 50 kg; and
c. 300 mg for patients of body weight category III, having a bodyweight above 50 kg.

In one embodiment, the maintenance dose of the antibody for use according to any of the above described embodiments of the fifth aspect of the disclosure is administered to patients of:

a) body weight category I in form of a single injection of 0.9 ml; and
b) body weight category II in form of a single injection of 1.3 ml; or
c) body weight category III in form of a single injection of 2 ml or 2 injections of 1 ml.

In an additional embodiment, the antibody for use according to any of the above described embodiments of the fifth aspect of the disclosure is used in a treatment that lasts up to 52 weeks after day 1.

In an additional embodiment, the antibody for use according to any of the above described embodiments of the fifth aspect of the disclosure is administered to patients of an age ranging between 6 and 21 years.

In one embodiment, the antibody for use according to any of the above described embodiments of the fifth aspect of the disclosure is selected from the group consisting of:

a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

In one additional embodiment, the antibody for use according to any of the above described embodiments of the fifth aspect of the disclosure comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In an additional embodiment, the antibody for use according to any of the above-described embodiments of the fifth aspect of the disclosure is CFZ533.

In another embodiment, the antibody for use according to any of the above-described embodiments of the fifth aspect of the disclosure is administered to the patients within the first 100 days after diagnosis of insulitis in said patient.

In another embodiment, the antibody for use according to any of the above-described embodiments of the fifth aspect of the disclosure is administered to the patients within the first 100 days after diagnosis of T1DM in said patient, but not before two weeks (14 days) after the diagnosis.

A sixth aspect of the disclosure relates to a pharmaceutical composition comprising a therapeutically effective amount of the antibody for use according to any of the above described embodiments of the fifth aspect of the disclosure and one or more pharmaceutically acceptable carriers.

In a seventh aspect of the disclosure a method of treating insulitis in a human subject is provided, comprising administering a therapeutically effective dose of an anti-CD40 antibody with silenced ADCC activity to said subject, wherein said antibody is administered through a loading dosing followed by a maintenance dosing and the route of administration is subcutaneous or intravenous, or a combination of subcutaneous or intravenous.

In an additional embodiment, the method according to the seventh aspect of the disclosure comprises a loading dosing of a first dose of the anti-CD40 antibody that is administered via intravenous injection and a maintenance dosing of the anti-CD40 antibody of a second dose that is administered via subcutaneous injections, wherein the second dose is different from the first dose.

In one embodiment, the method according to any of the above described embodiments of the seventh aspect of the disclosure comprises a loading dose of the anti-CD40 antibody of about 3 mg to about 60 mg antibody per kilogram of the patient.

In one embodiment, the method according to any of the above described embodiments of the seventh aspect of the disclosure comprises a loading dose of the anti-CD40 antibody of about 10 mg to about 30 mg antibody per kilogram of the patient.

In another embodiment, the patient treated according to the method of any of the above-described embodiments of the seventh aspect of the disclosure is a pediatric patient.

In an additional embodiment, the method according to any of the above-described embodiments of the seventh aspect of the disclosure comprises administration of a loading dose of 30 mg/kg of the anti-CD40 antibody administered intravenously at day 1 and administration of a maintenance dose of the anti-CD40 antibody administered subcutaneously as a fixed dose between 100 mg-350 mg once weekly starting at day 8.

In another embodiment, the method according to any of the above-described embodiments of the seventh aspect of the disclosure comprises subcutaneous administration of a maintenance dose of the anti-CD40 antibody as a fixed dose by body weight once weekly starting at day 8 at a dose of:

a. 135 mg for patients of body weight category I, having a bodyweight between 20 to 30 kg;
b. 195 mg for patients of body weight category II, having a bodyweight between 30 to 50 kg; and
c. 300 mg for patients of body weight category III, having a bodyweight above 50 kg.

In an additional embodiment, the method according to any of the above described embodiments of the seventh aspect of the disclosure comprises administration of the anti-CD40 antibody to the patients of the

a) body weight category I in form of a single injection of 0.9 ml; and
b) body weight category II in form of a single injection of 1.3 ml; or
c) body weight category III in form of a single injection of 2 ml or 2 injections of 1 ml.

In another embodiment, the method according to any of the above-described embodiments of the seventh aspect of the disclosure comprises a treatment of the patients with the anti-CD40 antibody for up to 52 weeks after day 1.

In one embodiment, the age range of the patients treated using the method according to any of the above-described embodiments of the seventh aspect is between 6 and 21 years.

In another embodiment, the anti-CD40 antibody used in the method according to any of the above described embodiments of the seventh aspect of the disclosure is selected from the group consisting of:

a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

In an additional embodiment, the anti-CD40 antibody used in the method according to any of the above described embodiments of the seventh aspect of the disclosure comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In one embodiment, the anti-CD40 antibody used in the method according to any of the above-described embodiments of the seventh aspect of the disclosure is CFZ533.

In another embodiment, the antibody used in the method according to any of the above-described embodiments of the seventh aspect of the disclosure is administered to the patients within the first 100 days after diagnosis of insulitis in said patient.

In another embodiment, the antibody used in the method according to any of the above-described embodiments of the seventh aspect of the disclosure is administered to the patients within the first 100 days after diagnosis of T1DM in said patient, but not before two weeks (14 days) after the diagnosis.

An eights aspect of the disclosure relates to the use of a liquid pharmaceutical composition comprising an anti-CD40 antibody with silenced ADCC activity, a buffer, a stabilizer and a solubilizer, for the manufacture of a medicament for the treatment of insulitis, wherein the anti-CD40 antibody is selected from the group consisting of:

i. an anti-CD40 antibody comprising an immunoglobulin VH domain comprising the amino acid sequence of SEQ ID NO: 7 and an immunoglobulin VL domain comprising the amino acid sequence of SEQ ID NO: 8;
ii. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
iii. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
iv. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14; and v. an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In an additional embodiment the eights aspect of the disclosure relates to the use of a liquid pharmaceutical composition comprising an anti-CD40 antibody, a buffer, a stabilizer and a solubilizer, for the manufacture of a medicament for the treatment of insulitis, wherein the anti-CD40 antibody:

a. is to be intravenously administered with a first loading dosing; and
b. thereafter, with a second maintenance dosing regimen administered subcutaneously, wherein said anti-CD40 antibody is selected from the group consisting of:
i. an anti-CD40 antibody comprising an immunoglobulin VH domain comprising the amino acid sequence of SEQ ID NO: 7 and an immunoglobulin VL domain comprising the amino acid sequence of SEQ ID NO: 8;
ii. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
iii. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
iv. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14; and
v. an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In another embodiment, the antibody used in the manufacture of a medicament according to any of the above-described embodiments of the eights aspect of the disclosure is administered in a first loading dose to the patients in within the first 100 days after diagnosis of insulitis in said patient.

In another embodiment, the antibody used in the manufacture of a medicament according to any of the above-described embodiments of the eights aspect of the disclosure is administered to the patients within the first 100 days after diagnosis of T1DM in said patient, but not before two weeks (14 days) after the diagnosis.

### BRIEF DESCRIPTION OF THE FIGURES

FIG. 1 is a schematic representation of the study design of the first and a second cohort of the proof of concept study for CFZ533 in T1DM patients (CCFZ533X2207).

FIG. 2 is a predicted CFZ533 plasma concentrations by body weight category predicted PK profiles in body weight (BW) Category I (≥20 to <30 kg), Category II (≥30 to <50 kg), and Category III (≥50 kg). In each category, body weights were uniformly sampled. The red line is the population prediction (the median of individual predictions), the shaded area covers 90% of the population (5th and 95th percentiles for individual predictions). The subcutaneous bioavailability of CFZ533 in T1DM patients was estimated using PK data from healthy volunteers (CCFZ533X2101).

FIG. 3 is a graph showing predicted steady state trough plasma CFZ533 concentrations for T1DM subjects in body weight Category III (similar CFZ533 plasma Ctrough, ss values are predicted for all 3 body weight categories), compared to CFZ533 trough concentrations observed in ongoing or completed clinical studies with CFZ533 The predicted trough steady state plasma CFZ533 concentrations (Ctrough,ss) for T1DM patient/subjects in body weight (BW) Category III (median of 222 μg/mL; 90% of the population between 140-344 μg/mL; similar Ctrough,ss values predicted for body weight Categories I and II) are presented together with observed (and mean) trough concentrations from ongoing (preliminary data) or completed clinical trials.

(i) Ctrough at Day 141 in Study CCFZ533X2204 (IV) in Myasthenia Gravis patients, (ii) and (iii) Ctrough at Day 29 and Day 85 in Study CCFZ533X2205 (IV) in Graves' Disease patients, respectively, (iv) Ctrough at Day 113 in Study CCFZ533X2203-Cohort 2 (IV), (v) and (vi) Ctrough at Day 85 in Study CCFZ533X2203-Cohort 3 Arm 2 (IV/SC) and Cohort 3 Arm 1 (SC/SC), in Primary Sjögren's Syndrome patients, respectively, and (vii) Ctrough at Day 337 in Study CCFZ533X2201-Part 2 (IV) in kidney transplant patients.

FIG. 4 is graphs showing in vitro CFZ533 inhibition of the rCD154-induced pathway activation.

FIG. 5 is graphs showing CFZ533 minimal stimulatory activity in vitro.

FIG. 6 is graphs showing that CFZ533 does not mediate cell depletion in vitro.

FIG. 7 is representative images of individual RI-1 B cells showing internalization of CD40 receptors upon binding by recCD154 or CFZ533.

FIG. 8 is graphs showing the pharmacokinetic and pharmacodynamic (target engagement; no B cell depletion) properties of CFZ533 in non-human primates.

FIG. 9A is an experimental design schematic of a PK/PD and vaccination study in non-human primates. FIG. 9B is graphs showing anti-KLH IgG (immune response) and plasma CFZ533 levels (pharmacokinetics). FIG. 9C shows results of a histological analysis of germinal centers.

FIG. 10 shows experimental results; CD40 signature in ELS in salivary glands and reduction of tertiary lymphoid organs in salivary glands of NOD mice after 10-week treatment with MR1.

FIG. 11 shows experimental results; Increased percentage of AQP-5-positive cells in salivary glands of NOD mice after 10-week treatment with anti-CD154.

### DETAILED DESCRIPTION OF THE DISCLOSURE

Without wishing to be bound by theory, the inventors have identified that sustained plasma concentrations of at least about 40 μg/mL of an anti-CD40 antibody with silenced ADCC activity (antibody with silenced antibody-dependent cell mediated cytotoxicity; Borrok et al., 2017; e.g. the CFZ533 antibody) during the maintenance regimen was necessary to block the CD40-CD40L pathway in target tissues in patients suffering from an autoimmune disease, such as primary Sjogren's syndrome patients (PCT/IB2018/058537, unpublished)

CD40 plays a pathogenic role in T1DM and insulitis, and disrupting CD40:CD154 interactions or activity is envisaged by the inventors to be an effective autoimmune therapeutic strategy for this disorder. T1DM develops when pathogenic autoreactive immune cells invade the pancreatic islets, and damage insulin producing β-cells (Katsarou et al 2017). Insulitis is a hallmark of aggressive disease and has higher prevalence in pediatric patients (Leete et al 2016).

CD40 is a transmembrane glycoprotein in the tumor necrosis factor receptor superfamily expressed by immune and non-immune cells. CD154, the ligand for CD40 is also widely expressed (Peters et al 2009). CD40:CD154 interactions mediate T-dependent B cell responses and is important for priming and activation of CD4+ autoreactive T lymphocytes and CD8+ cytolytic T lymphocytes.

CD40 is upregulated upon antigen presenting cell (APC) activation, and the interaction between CD40+ APCs such as B lymphocytes with naïve T lymphocytes induces the production of CD154 on the surface of these lymphocytes. CD40 signaling may also function at the level of T cell selection in the thymus, and enhance production of pro-inflammatory cytokines, which can further influence T cell differentiation into active Th17 cells (Iezzi et al 2009). Non-clinical data support a role for the CD40:CD154 costimulatory pathway in defective tolerance in autoreactive T lymphocytes, development of insulitis and diabetes (Price et al 2014). Data in the non-obese diabetic (NOD) mouse, a model of spontaneous autoimmune diabetes, supports a causative and functional role of the CD40:CD154 pathway in the initiation of insulitis and diabetes in the NOD mouse (Balasa et al 1997; Eshima et al 2003, Vaitaitis et al 2014, Vaitaitis et al 2017).

Clinical data in patients with T1DM also support a role for the CD40-CD154 costimulatory pathway disease pathogenesis. Pediatric patients with new onset T1DM have 2-fold elevated levels of soluble CD40 (sCD40) (Chatzigeorgiou et al 2010b). T1DM patients with disease duration from diagnosis between 1 and 6 months exhibit the highest levels of sCD40. Elevated levels of sCD40 in pediatric patients with new onset T1DM are positively associated with HbA1c, hyperglycemia, and inflammatory markers, CRP, IL-6 and MMP-9 (Chatzigeorgiou et al 2010b, Chatzigeorgiou et al 2010a). Together these observations point to a role for the CD40:CD154 pathway in T1DM in general and in pediatric new onset T1DM that could respond to an intervention with an anti-CD40 monoclonal antibody with silenced ADCC activity like CFZ533, provided that the antibody is administered in accordance with the herein disclosed treatment and dosage regimen.

Pediatric compared to adults patients are at increased risk for acute complications of T1DM, diabetic ketoacidosis and severe hypoglycemia (a side effect of insulin); and for death from these complications (Wherrett et al 2015). Pediatric patients are also at higher risk for neurocognitive changes due to chronic hyperglycemia and severe hypoglycemia (Wherrett et al 2015). Transformative therapies are urgently needed to improve clinical outcomes for children with T1DM.

It is therefore envisaged by the inventors that blockade of CD40-CD154 activation by applying the herein disclosed treatment regimen, resulting in a sustained and pharmacologically effective plasma concentrations of an anti-CD40 antibody with silenced ADCC activity (e.g. CFZ533 antibody), could halt the immune-mediated β-cell destruction and the insulitis resulting in preservation of residual β-cell function.

It is furthermore envisaged by the inventors that the provision of a sustained and pharmacologically effective plasma concentrations of an anti-CD40 antibody with silenced ADCC activity (e.g. CFZ533 antibody), as disclosed herein, may be in particular effective in the treatment of patients with new-onset of T1DM and/or insulitis.

Thus, any anti-CD40 antibody capable of blocking CD40-CD154 signaling with silenced ADCC activity, could be suitable for the treatment of T1DM and/or insulitis.

Also, because CFZ533 is subjected to target mediated disposition (which is in relation to target turnover and expression), and T1DM patients are presenting with high CD40 expression in the body, a loading regimen will be necessary at start of treatment to fully saturate CD40 receptors in these patients in conditions where CD40 levels have been enhanced, requiring higher doses or a more frequent regimen at start of treatment. Thus, with a loading dosing regimen providing at start of treatment rapid saturation of CD40 receptors, followed by a maintenance dosing regimen providing, throughout the entire treatment period, sustained plasma concentrations of at least 40 μg/mL, in situations where CD40 expression in affected tissues would be enhanced (severity of the condition), is considered for a therapeutic effect. In previous studies with anti-CD40 antibody with silenced ADCC activity, e.g. CFZ533, the observed maximum plasma concentration at steady state was between about 300 and 400 μg/mL (Cohort 3; Study NCT02291029; CCFZ533X2203) and was generally safe and well tolerated, with no major signal to suggest increased risk of infection. No thromboembolic event observed. Hence, it is envisaged by the inventors that the herein disclosed treatment methods for the first time provide an efficacious and safe treatment of T1DM and/or insulitis, in particular for pediatric patients at the onset of T1DM and/or insulitis.

The appropriate dosage will vary depending upon, for example, the particular anti-CD40 antibody or antigen binding fragment thereof (e.g., mAb1, also called CFZ533 herein, mAb2 or ASKP1240) to be employed, the age and weight of the patient, the history of the disease (e.g. T1DM disease onset and progression) and the nature and severity of the condition being treated (e.g. insulitis). Ultimately, the attending health care provider will decide the amount of the anti-CD40 antibody with silenced ADCC activity studies with which to treat each individual patient in order to achieve the pharmacologically effective plasma concentrations as described herein. In some embodiments, the attending health care provider may administer low doses of the anti-CD40 antibody with silenced ADCC activity and observe the patient's response. In other embodiments, the initial dose(s) of anti-CD40 antibody with silenced ADCC activity administered to a patient are high, and then are titrated downward until signs of relapse occur. Larger doses of the anti-CD40 antibody with silenced ADCC activity may be administered until the optimal therapeutic effect is obtained for the patient, and the dosage is not generally increased further.

In practicing some of the envisaged methods of treatment or uses of the present disclosure, a therapeutically effective amount of an anti-CD40 antibody with silenced ADCC activity, (e.g., mAb1, also called CFZ533 herein, mAb2, ASKP1240) or antigen-binding fragment thereof is administered to a patient, e.g., a mammal (e.g., a human).

Typically, antibodies or proteins are administered by injection, for example, either intravenously, intraperitoneally, or subcutaneously. Methods to accomplish this administration are known to those of ordinary skill in the art. As will be appreciated by a person skilled in the art, any suitable means for administering can be used, as appropriate for a particular selected route of administration.

Examples of possible routes of administration include parenteral, (e.g., intravenous (i.v., I.V. or IV), intramuscular, intradermal, subcutaneous (s.c., S.C. or SC), or infusion), oral and pulmonary (e.g., inhalation), nasal, transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

An advantage of having a therapeutic regimen divided into a loading dosing part and a maintenance dosing part is that it allows for optimal therapeutic effect. For all therapeutic regimens described herein, the purpose of the loading dosing is to achieve target saturation (plasma concentrations at least close to 40 μg/mL) and thus onset of therapeutic effect, and the purpose of the maintenance dosing is to sustain efficacy.

An anti-CD40 antibody therapy as described herein can be initiated by administering a loading regimen or loading dosing of the antibody or antigen binding fragment thereof to the subject in need of anti-CD40 antibody therapy. By “loading dose(s)” is intended an initial dosing of the anti-CD40 antibody or antigen binding fragment thereof that is administered to the subject one or several times, where the dose of the antibody or antigen binding fragment thereof administered falls within the higher dosing range (i.e., from about 10 mg/kg to about 60 mg/kg, such as about 30 mg/kg intravenously, or about 600 mg, or about 300 mg or about 150 mg weekly, bi-weekly for up to 4 weeks). The “loading regimen” can be administered as a single administration or multiple administrations, for example, a single or multiple intravenous infusion(s), or as multiple subcutaneous administrations combined in a “loading dosing” regimen depending on the severity of the disease). Following administration of the “loading regimen”, the subject is then administered one or more additional therapeutically effective doses of the anti-CD40 antibody or antigen binding fragment thereof (maintenance dosing/regimen). Subsequent therapeutically effective maintenance doses can be administered, for example, according to a weekly dosing schedule, or once every two weeks (bi-weekly), once every three weeks, or once every four weeks. In such embodiments, the subsequent therapeutically effective doses generally fall within the lower dosing range (i.e. about 0.3 mg/kg to about 30 mg/kg, such as about 10 mg/kg, e.g. 10 mg/kg IV or about 150 mg, about 300 mg or about 600 mg administered weekly, bi-weekly or every 4 weeks subcutaneously).

Alternatively, in some embodiments, following the “loading regimen”, the subsequent therapeutically effective doses of the anti-CD40 antibody or protein are administered according to a “maintenance schedule”, wherein the therapeutically effective dose of the antibody or protein is administered once a week or once a month, wherein the treatment can continue up to 6 weeks, 10 weeks, three months, four months, five months, six months, etc. up to 12 months or more.

The timing of dosing is generally measured from the day of the first dose of the active compound (e.g., mAb1), which is also known as “baseline”. However, different health care providers use different naming conventions.

Notably, week zero may be referred to as week 1 by some health care providers, while day zero may be referred to as day one by some health care providers. Thus, it is possible that different physicians will designate, e.g., a dose as being given during week 3/on day 21, during week 3/on day 22, during week 4/on day 21, during week 4/on day 22, while referring to the same dosing schedule. For consistency, the first week of dosing will be referred to herein as week 0, while the first day of dosing will be referred to as day 1. However, it will be understood by a skilled artisan that this naming convention is simply used for consistency and should not be construed as limiting, i.e., weekly dosing is the provision of a weekly dose of the anti-CD40 antibody, e.g., mAb1, regardless of whether the physician refers to a particular week as “week 1” or “week 2”. It will be understood that a dose need not be provided at an exact time point, e.g., a dose due approximately on day 29 could be provided, e.g., on day 24 to day 34, e.g., day 30, as long as it is provided in the appropriate week.

Thus, an anti-CD40 antibody with silenced ADCC activity for use in the treatment of T1DM or insulitis can be administered in different ways, e.g. through a loading dosing followed by a maintenance dosing and the route of administration is subcutaneous or intravenous, or a combination of subcutaneous or intravenous. In one embodiment, the loading dose of the anti-CD40 antibody with silenced ADCC activity is administered via intravenous injection and the maintenance dosing is administered via subcutaneous injections.

In another particular embodiment, the intravenously administered loading dose of the anti-CD40 antibody with silenced ADCC activity is a first dose and the subcutaneously administered maintenance dose is a second dose being different from the first dose.

While it is understood that the disclosed methods provide for treatment of T1DM and/or insulitis patients using an anti-CD40 antibody with silenced ADCC activity (e.g., mAb1/CFZ533, mAb2, ASKP1240), this does not preclude that, if the patient is to be ultimately treated with such anti-CD40 antibody, the therapy is necessarily a monotherapy. Indeed, if a patient is selected for treatment with an anti-CD40 antibody with silenced ADCC activity, then the anti-CD40 antibody with silenced ADCC activity (e.g., mAb1/CFZ533, mAb2, ASKP1240) may be administered in accordance with the methods of the disclosure either alone or in combination with other agents and therapies.

It will be understood that regimen changes may be appropriate for certain T1DM or insulitis patients e.g., patients that display inadequate response to treatment with the anti-CD40 antibody or antigen binding fragment thereof (e.g., mAb1, also called CFZ533 herein, mAb2, ASKP1240). Thus, administration (e.g. mAb1/CFZ533 or mAb2) to achieve the herein described pharmacologically effective plasma concentrations may be more frequent than weekly dosing.

Some patients may benefit from a loading regimen (e.g., weekly administrations for several weeks [e.g., 1 to 4 weeks, e.g., dosing at weeks 0, 1, 2, and/or 3, such as 2 weeks, loading dosing regimen at weeks 0 and 1] followed by a maintenance regimen starting e.g. at week 3 or 4 where the anti-CD40 antibody with silenced ADCC activity (e.g., mAb1/CFZ533, mAb2, ASKP1240) may be administered weekly, bi-weekly or every 4 weeks for several weeks. It is understood that the route of administration (e.g. subcutaneous vs. intravenous) and the injection volume needed to achieve the herein described specific plasma concentrations of the anti-CD40 antibody may require adaptation of the dosing regimen.

For example, an appropriate regimen for mAb1/CFZ533 or mAb2 can be weekly for several weeks [e.g., 1 to 4 weeks, e.g., dosing at weeks 0, 1, 2, and 3] followed by a monthly maintenance regimen.

In another example, an appropriate regimen for mAb1/CFZ533 or mAb2 can be weekly for several weeks (e.g., 2 to 8 weeks, such as 3 weeks, e.g., dosing at weeks 0, 1, 2) followed by a bi-weekly maintenance regimen.

It will also be understood that administration (e.g. for mAb1/CFZ533 or mAb2) may be less frequent than monthly dosing, e.g., dosing every 6 weeks, every 8 weeks (every two months), quarterly (every three months), etc.

It will be understood that dose escalation may be appropriate for certain T1DM or insulitis patients, based on severity of the disease, e.g., patients that display inadequate response to treatment with the CD40 pathway antagonists, e.g. an anti-CD40 antibody or antigen binding fragment thereof (e.g., mAb1, also called CFZ533 herein, mAb2, ASKP1240). Thus, subcutaneous (s.c.) dosages (loading or maintenance doses) may be greater than about 50 mg s.c., e.g., about 75 mg, about 100 mg, about 125 mg, about 175 mg, about 200 mg, about 250 mg, about 350 mg, about 400 mg, about 450 mg, about 500 mg, about 600 mg, etc.; similarly, intravenous (i.v.) dosages (loading or maintenance doses) may be greater than about 10 mg/kg, e.g., about 11 mg/kg, 12 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg etc. It will also be understood that dose reduction may also be appropriate for certain T1DM ands/or insulitis patients, e.g., patients that display adverse events or an adverse response to treatment with the Anti-CD40 antibody with silenced ADCC activity

The treatment regimens described in the following are believed to (i) rapidly saturate CD40 receptors in target tissues (i.e. pancreatic lymph nodes) and to minimize the CD40-mediated elimination of CFZ533 in conditions where the aggressiveness of the disease within 100 days of diagnosis (insulitis, B- and T-lymphocytes infiltration in pancreatic islets, active ectopic germinal centers) is likely to be associated with high tissue CD40 expression, and to (ii) rapidly block the aggressive autoimmune destruction of residual β-cells, insulitis and local infiltration of pathogenic auto reactive B lymphocytes. Hence, in a specific embodiment of the disclosure, the anti-CD40 antibody with silenced ADCC activity or an antigen binding fragment thereof (e.g., mAb1, also called CFZ533 herein, mAb2, ASKP1240) is administered to the patient within the first 100 days after said patient has been diagnosed with T1DM or insulitis.

In some embodiments, the anti-CD40 antibody with silenced ADCC activity or an antigen binding fragment thereof (e.g., mAb1, also called CFZ533 herein, mAb2, ASKP1240) may be administered to the patient at a body weight-adjusted intravenous (IV) loading dose between 3 mg/kg to 60 mg/kg on Day 1 (Week 0), e.g., about 3 mg/kg, about 4 mg/kg, about 5 mg/kg, about 6 mg/kg, about 7 mg/kg, about 8 mg/kg, about 9 mg/kg, about 10 mg/kg, 11 mg/kg, 12 mg/kg, 15 mg/kg, 20 mg/kg, 25 mg/kg, 30 mg/kg, 35 mg/kg, 40 mg/kg, 45 mg/kg, 50 mg/kg, 55 mg/kg or 60 mg/kg, followed by a herein disclosed maintenance dosage regimen, e.g. maintenance dose regimen I, II, III or IV. In one embodiment, the anti-CD40 antibody with silenced ADCC activity is administered to the patient at a body weight-adjusted intravenous (IV) loading dose between 3 mg/kg to 30 mg/kg on Day 1 (Week 0), followed by a herein disclosed maintenance dosage regimen, e.g. maintenance dose regimen I, II, III or IV.

In one embodiment, the anti-CD40 antibody with silenced ADCC activity is administered to the patient at a body weight-adjusted intravenous (IV) loading dose between 10 mg/kg to 30 mg/kg on Day 1 (Week 0), followed by a herein disclosed maintenance dosage regimen, e.g. maintenance dose regimen I, II, III or IV.

In one embodiment, the anti-CD40 antibody with silenced ADCC activity is administered to the patient at a body weight-adjusted intravenous (IV) loading dose of 10 mg/kg or 30 mg/kg on Day 1 (Week 0), followed by a herein disclosed maintenance dosage regimen. In a particularly specified embodiment the maintenance dose is administered s.c. to the patients once weekly up to week 52.

In one specific embodiment, 3 mg/kg CFZ533 is administered i.v. on day 1 (D1) followed by a herein disclosed maintenance dosage regimen, e.g. maintenance dose regimen I, II, III or IV.

In a specific embodiment, 10 mg/kg CFZ533 is administered i.v. on day 1 (D1), followed by a herein disclosed maintenance dosage regimen, e.g. maintenance dose regimen I, II, III or IV.

In another specific embodiment, 30 mg/kg CFZ533 is administered i.v. on D1 followed by a herein disclosed maintenance dosage regimen, e.g. maintenance dose regimen I, II, III or IV.

In another specific embodiment, 40 mg/kg CFZ533 is administered i.v. on D1 followed by a herein disclosed maintenance dosage regimen, e.g. maintenance dose regimen I, II, III or IV.

In another specific embodiment, 50 mg/kg CFZ533 is administered i.v. on D1 followed by a herein disclosed maintenance dosage regimen, e.g. maintenance dose regimen I, II, III or IV.

In another specific embodiment, 60 mg/kg CFZ533 is administered i.v. on D1 followed by a herein disclosed maintenance dosage regimen, e.g. maintenance dose regimen I, II, III or IV.

Therefore, in one embodiment, the anti-CD40 antibody with silenced ADCC activity (e.g., mAb1, also called CFZ533) or an antigen-binding fragment thereof is administered to the patient at an initial dose of 10 mg/kg or 30 mg/kg delivered i.v., and the maintenance dose is then adjusted to a fixed dose between 100 mg and 350 mg delivered s.c. once weekly starting at day 8 (maintenance dose regimen I).

In some embodiments, the anti-CD40 antibody with silenced ADCC activity (e.g., mAb1, also called CFZ533) or an antigen-binding fragment thereof may be administered to the patient at an initial dose of 10 mg/kg or 30 mg/kg delivered i.v. on D1, and the maintenance dose is adjusted to a fixed dose between 100 mg and 350 mg delivered s.c. once weekly, wherein pediatric patients shall be dosed once weekly starting at D1 according to the following body weight categories fixed maintenance dose:

i. Body weight Category I (≥20 to <30 kg): between 100-150 mg,
ii. Body weight Category II (≥30 to <50 kg): between 150-250 mg,
iii. Body weight Category III (≥50 kg): between 250-350 mg (jointly maintenance dose regimen II).

In a specific embodiment, the weekly s.c. maintenance dose of between 100-150 mg, 150-250 mg or 250-350 mg administered to the patients of the Body weight classes I-III will be defined based on the body weight of the subject recorded every 3 months on Day 1/Week 0, Day 85/Week 12, Day 169/Week 24, Day 253/Week 36 and Day 337/Week 48 to account for body weight gain or loss during the treatment period.

In yet another specific embodiment, a loading dose which comprises one dose of 10 mg/kg CFZ533 administered i.v. on D1, is followed by a maintenance dose which comprises unit doses of 100-350 mg administered s.c. once weekly (Q1W), i.e. 300 mg CFZ533 s.c. once weekly from D8.

In yet another specific embodiment, a loading dose which comprises one dose of 10 mg/kg CFZ533 is administered i.v. to the patient on D1, is followed by a maintenance dose s.c. once weekly (Q1W) from D8, which comprises the following body weight categories fixed dose:

i. Body weight Category I (≥20 to <30 kg): between 100-150 mg,

ii. Body weight Category II (≥30 to <50 kg): between 150-250 mg,

iii. Body weight Category III (≥50 kg): between 250-350 mg.

In yet another specific embodiment, a loading dose which comprises one dose of 10 mg/kg CFZ533 administered i.v. on D1, is followed by a maintenance dose s.c. once weekly (Q1W) from D8, which comprises the following body weight categories fixed dose:

i. Body weight Category I (≥20 to <30 kg): between 135 mg,

Body weight Category II (≥30 to <50 kg): between 195 mg,

Body weight Category III (≥50 kg): between 300 mg (jointly maintenance dose regimen III).

In yet another specific embodiment, a loading dose which comprises one dose of 20 mg/kg CFZ533 is administered i.v. on D1, is followed by a maintenance dose which comprises unit doses of 100-350 mg administered s.c. once weekly (Q1W), i.e. 300 mg CFZ533 s.c. once weekly from D8.

In yet another specific embodiment, a loading dose which comprises one dose of 20 mg/kg CFZ533 is administered i.v. on D1, is followed by a maintenance dose s.c. once weekly (Q1W) from D8, which comprises the following body weight categories fixed dose:

iv. Body weight Category I (≥20 to <30 kg): between 100-150 mg,

v. Body weight Category II (≥30 to <50 kg): between 150-250 mg,

vi. Body weight Category III (≥50 kg): between 250-350 mg.

In yet another specific embodiment, a loading dose which comprises one dose of 20 mg/kg CFZ533 administered i.v. on D1, is followed by a maintenance dose s.c. once weekly (Q1W) from D8, which comprises the following body weight categories fixed dose:

ii. Body weight Category I (≥20 to <30 kg): between 135 mg,

iii. Body weight Category II (≥30 to <50 kg): between 195 mg,

iv. Body weight Category III (≥50 kg): between 300 mg.

In yet another specific embodiment, a loading dose which comprises one dose of 30 mg/kg CFZ533 administered i.v. on D1, is followed by a maintenance dose which comprises unit doses of 100-350 mg administered s.c. once weekly (Q1W), i.e. 300 mg CFZ533 s.c. once weekly from D8.

In yet another specific embodiment, a loading dose which comprises one dose of 30 mg/kg CFZ533 is administered i.v. on D1, is followed by a maintenance dose s.c. once weekly (Q1W) from D8, which comprises the following body weight categories fixed dose:

i. Body weight Category I (≥20 to <30 kg): between 100-150 mg,

ii. Body weight Category II (≥30 to <50 kg): between 150-250 mg,

iii. Body weight Category III (≥50 kg): between 250-350 mg.

In yet another specific embodiment, a loading dose which comprises one dose of 30 mg/kg CFZ533 administered i.v. on D1, is followed by a maintenance dose s.c. once weekly (Q1W) from D8, which comprises the following body weight categories fixed dose:

i. Body weight Category I (≥20 to <30 kg): between 135 mg,

ii. Body weight Category II (≥30 to <50 kg): between 195 mg,

iii. Body weight Category III (≥50 kg): between 300 mg.

In yet another specific embodiment, a loading dose which comprises one dose of 40 mg/kg CFZ533 administered i.v. on D1, is followed by a maintenance dose which comprises unit doses of 100-350 mg administered s.c. once weekly (Q1W), i.e. 300 mg CFZ533 s.c. once weekly from D8.

In yet another specific embodiment, a loading dose which comprises one unit dose of 40 mg/kg CFZ533 is administered i.v. on D1, is followed by a maintenance dose s.c. once weekly (Q1W) from D8, which comprises the following body weight categories fixed dose:

i. Body weight Category I (≥20 to <30 kg): between 100-150 mg,

ii. Body weight Category II (≥30 to <50 kg): between 150-250 mg,

iii. Body weight Category III (≥50 kg): between 250-350 mg.

In yet another specific embodiment, a loading dose which comprises one dose of 40 mg/kg CFZ533 administered i.v. on D1, is followed by a maintenance dose s.c. once weekly (Q1W) from D8, which comprises the following body weight categories fixed dose:

i. Body weight Category I (≥20 to <30 kg): between 135 mg,

ii. Body weight Category II (≥30 to <50 kg): between 195 mg,

iii. Body weight Category III (≥50 kg): between 300 mg.

In yet another specific embodiment, a loading dose which comprises one dose of 50 mg/kg CFZ533 administered i.v. on D1, is followed by a maintenance dose which comprises unit doses of 100-350 mg administered s.c. once weekly (Q1W), i.e. 300 mg CFZ533 s.c. once weekly from D8.

In yet another specific embodiment, a loading dose which comprises one dose of 50 mg/kg CFZ533 is administered i.v. on D1, is followed by a maintenance dose s.c. once weekly (Q1W) from D8, which comprises the following body weight categories fixed dose:

i. Body weight Category I (≥20 to <30 kg): between 100-150 mg,

ii. Body weight Category II (≥30 to <50 kg): between 150-250 mg,

iii. Body weight Category III (≥50 kg): between 250-350 mg.

In yet another specific embodiment, a loading dose which comprises one dose of 50 mg/kg CFZ533 administered i.v. on D1, is followed by a maintenance dose s.c. once weekly (Q1W) from D8, which comprises the following body weight categories fixed dose:

i. Body weight Category I (≥20 to <30 kg): between 135 mg,

ii. Body weight Category II (≥30 to <50 kg): between 195 mg,

iii. Body weight Category III (≥50 kg): between 300 mg.

In yet another specific embodiment, a loading dose which comprises one dose of 60 mg/kg CFZ533 administered i.v. on D1, is followed by a maintenance dose which comprises unit doses of 100-350 mg administered s.c. once weekly (Q1W), i.e. 300 mg CFZ533 s.c. once weekly from D8.

In yet another specific embodiment, a loading dose which comprises one dose of 60 mg/kg CFZ533 is administered i.v. on D1, is followed by a maintenance dose s.c. once weekly (Q1W) from D8, which comprises the following body weight categories fixed dose:

i. Body weight Category I (≥20 to <30 kg): between 100-150 mg,

ii. Body weight Category II (≥30 to <50 kg): between 150-250 mg,

iii. Body weight Category III (≥50 kg): between 250-350 mg.

In yet another specific embodiment, a loading dose which comprises one dose of 60 mg/kg CFZ533 administered i.v. on D1, is followed by a maintenance dose s.c. once weekly (Q1W) from D8, which comprises the following body weight categories fixed dose:

i. Body weight Category I (≥20 to <30 kg): between 135 mg,

ii. Body weight Category II (≥30 to <50 kg): between 195 mg,

iii. Body weight Category III (≥50 kg): between 300 mg.

In a particular embodiment the maintenance dose which comprises unit doses of between 100-150 mg (Body weight Category I), between 150-250 mg (Body weight Category II) or between 250-350 mg (Body weight Category III) is administered s.c. to the patients once weekly up to week 52.

In another embodiment the maintenance dose which comprises unit doses of 135 mg (Body weight Category I), 195 mg (Body weight Category II) or 300 mg (Body weight Category III) is administered s.c. to the patient once weekly from Day 8 up to week 52 (jointly maintenance dose regimen IV).

In on embodiment of the disclosure, the loading dosages are administered i.v. using a liquid pharmaceutical composition comprising the CFZ533 at a concentration of 100 mg/ml to 350 mg/ml. In a specified embodiment the liquid pharmaceutical composition for i.v. administration comprises CFZ533 at a concentration of 150 mg/mL or 300 mg/mL

In on embodiment of the disclosure, the maintenance dosages (e.g. maintenance dose regimens I-IV) are administered s.c. using a liquid pharmaceutical composition comprising CFZ533 at a concentration between 100 mg/ml to 350 mg/ml. In a specified embodiment the liquid pharmaceutical composition comprises CFZ533 at a concentration of 150 mg/mL or 300 mg/ml. From this follows that the following administration regimens are preferred for

a) Body weight Category I patients: 135 mg=single injection of 0.9 mL;

b) Body weight Category II patients: 195 mg=single injection of 1.3 mL;

c) Body weight Category III patients: 300 mg=1 injection of 2 mL or 2 injections of 1 mL).

The anti-CD40 antibody or antigen-binding fragment thereof may be CFZ533, a functional derivative thereof or a biosimilar thereof.

As herein defined, “unit dose” refers to a dose that can be comprised between about 75 mg to 900 mg, e.g. about 150 mg to about 600 mg, e.g. about 150 mg to about 600 mg, e.g. about 300 mg to about 600 mg, or a e.g. about 150 mg to about 300 mg of the drug substance. For example an unit s.c. dose is about 75 mg, about 150 mg, about 300 mg, about 350 mg, about 400 mg, about 450 mg, about 500 mg, about 550 mg, about 600 mg drug substance.

In a particular embodiment of the disclosure, the anti-CD40 antibody with silenced ADCC activity for use in the treatment of T1DM or insulitis is selected from the group consisting of:
a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

In yet another specific embodiment the anti-CD40 antibody with silenced ADCC activity for use in the treatment of T1DM or insulitis comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In another preferred embodiment the anti-CD40 antibody with silenced ADCC activity for use in the treatment of T1DM or insulitis is CFZ533.

### Pharmaceutical Compositions

Therapeutic antibodies are typically formulated either in aqueous form ready for administration or as lyophilisate for reconstitution with a suitable diluent prior to administration. An anti-CD40 antibody with silenced ADCC activity for use in accordance with the disclosed uses or treatments may be formulated either as a lyophilisate, or as an aqueous composition, for example in pre-filled syringes. The formulation is also called drug product (DP).

Suitable formulation can provide an aqueous pharmaceutical composition or a lyophilisate which can be reconstituted to give a solution with a high concentration of the antibody active ingredient and a low level of antibody aggregation for delivery to a patient. High concentrations of antibody are useful as they reduce the amount of material which must be delivered to a patient. Reduced dosing volumes minimize the time taken to deliver a fixed dose to the patient. Aqueous compositions with high concentration of anti-CD40 antibodies are particularly suitable for subcutaneous or intravenous administration.

The anti-CD40 antibody with silenced ADCC activity may be used as a pharmaceutical composition when combined with a pharmaceutically acceptable carrier. Such a composition may contain, in addition to an anti-CD40 antibody such as mAb1 or mAb2, carriers, various diluents, fillers, salts, buffers, stabilizers, solubilizers, and other materials well known in the art. The characteristics of the carrier will depend on the route of administration. The pharmaceutical compositions for use in the disclosed methods may also contain additional therapeutic agents for treatment of the particular targeted disorder.

In one specific embodiment the composition for use in the disclosed treatments and methods (drug product (DP)), is a lyophilized formulation prepared from an aqueous formulation having a pH of 6.0 and comprising:

(i) 150 mg/mL mAb1 or mAb2

(ii) 270 mM sucrose as a stabilizer,

(iii) 30 mM L-histidine as a buffering agent, and

(iv) 0.06% Polysorbate 20 as a surfactant.

In another specific embodiment the pharmaceutical composition (drug product (DP)), is an aqueous pharmaceutical composition has a pH of 6.0 and comprising:

(i) 150 mg/mL mAb1 or mAb2

(ii) 270 mM sucrose as a stabilizer,

(iii) 30 mM L-histidine as a buffering agent, and

(iv) 0.06% Polysorbate 20 as a surfactant.

Disclosed herein is the use of an anti-CD40 antibody (e.g., mAb1) for the manufacture of a medicament for the treatment of T1DM or insulitis in a patient, wherein the medicament is formulated to comprise containers, each container having a sufficient amount of the anti-CD40 antibody to allow delivery of at least about 75 mg, 150 mg, 300 mg or 600 mg anti-CD40 antibody or antigen binding fragment thereof (e.g., mAb1) per unit dose.

Disclosed herein is the use of an anti-CD40 antibody (e.g., mAb1) for the manufacture of a medicament for the treatment of T1DM in a patient, wherein the medicament is formulated at a dosage to allow systemic delivery (e.g., i.v. or s.c. delivery) of 75 mg, 150 mg, 300 mg of 600 mg anti-CD40 antibody or antigen binding fragment thereof (e.g., mAb1) per unit dose.

The disclosure also encompasses kits for treating a patient with T1DM or insulitis (as the case may be) with an anti-CD40 antibody with silenced ADCC activity or antigen binding fragment thereof, e.g., mAb1. Such kits comprise the anti-CD40 antibody or antigen binding fragment thereof, e.g., mAb1 (e.g., in liquid or lyophilized form) or a pharmaceutical composition comprising the anti-CD40 antibody. Additionally, such kits may comprise means for administering the anti-CD40 antibody (e.g., a syringe and vial, a prefilled syringe, a prefilled pen, a patch/pump) and instructions for use in T1DM or insulitis. The instructions may disclose providing the anti-CD40 antibody (e.g., mAb1) to the patient as part of a specific dosing regimen disclosed herein. These kits may also contain additional therapeutic agents for treating T1DM or insulitis, e.g., for delivery in combination with the enclosed anti-CD40 antibody, e.g., mAb1.

In one embodiment of the disclosure the kits for the treatment of a patient having T1DM or insulitis, comprising: a) a pharmaceutical composition comprising a therapeutically effective amount of an anti-CD40 antibody described herein or antigen binding fragment thereof, b) means for administering said anti-CD40 antibody or antigen binding fragment thereof to the patient; and c) instructions providing intravenously administering an anti-CD40 antibody described herein or antigen binding fragment thereof to a patient in need thereof as a loading dose of about 3 to about 60 mg active ingredient per kilogram of a human subject at D1 followed by once weekly subcutaneously dosing of the anti-CD40 antibody, e.g. in accordance with maintenance regimens I-IV disclosed herein.

In one specific embodiment, a use is provided of a) a liquid pharmaceutical composition comprising an anti-CD40 antibody, a buffer, a stabilizer and a solubilizer for the manufacture of a medicament for the treatment of T1DM or insulitis, wherein the anti-CD40 antibody:

is selected from the group consisting of:
a) an anti-CD40 antibody comprising an immunoglobulin VH domain comprising the amino acid sequence of SEQ ID NO: 7 and an immunoglobulin VL domain comprising the amino acid sequence of SEQ ID NO: 8;
b) an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c) an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
d) an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14; e) an anti-CD40 antibody comprising a silent Fc IgG1 region: and
f) an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

In one specific embodiment, a use is provided of a) a liquid pharmaceutical composition comprising an anti-CD40 antibody, a buffer, a stabilizer and a solubilizer for the manufacture of a medicament for the treatment of T1DM or insulitis, wherein the anti-CD40 antibody:

i) is to be intravenously administered to the patient with a dose of about 3 to about 30 mg, such as 10 mg, active ingredient per kilogram of a human subject at day 1, or two times, or three times once every other week; and

ii) thereafter, is to be subcutaneously administered to the patient as weekly fixed doses between 100 mg-350 mg once starting at day 8, wherein said anti-CD40 antibody is selected from the group consisting of:
g) an anti-CD40 antibody comprising an immunoglobulin VH domain comprising the amino acid sequence of SEQ ID NO: 7 and an immunoglobulin VL domain comprising the amino acid sequence of SEQ ID NO: 8;
h) an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
i) an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;
j) an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14;
k) an anti-CD40 antibody comprising a silent Fc IgG1 region: and
l) an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

### Definitions

As used herein, CD40 refers to cluster of differentiation 40, also called tumor necrosis factor receptor superfamily member 5. The term CD40 refers to human CD40, for example as defined in SEQ ID NO: 19, unless otherwise described.

The term “about” in relation to a numerical value×means, for example, +/−10%. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

The term “comprising” encompasses “including” as well as “consisting,” e.g., a composition “comprising” X may consist exclusively of X or may include something additional, e.g., X+Y.

AUC0-t designates the area under the plasma concentration-time curve from time zero to time ‘t’ where t is a defined time point after administration [mass×time/volume].

AUCtx-ty represents the area under the plasma concentration-time curve from time ‘x’ to time ‘y’ where ‘time x’ and ‘time y’ are defined time points after administration.

Cₘₐₓ is the observed maximum plasma concentration following drug administration [mass/volume].

Cₘᵢₙ is the observed minimum plasma concentration following drug administration C_{trough} is the observed plasma concentration that is just prior to the beginning of, or at the end of a dosing interval.

Tₘₐₓ is the time to reach the maximum concentration after drug administration [time]. ss (subscript) indicate that the parameter is defined at steady state.

The term “antibody” or “anti-CD40 antibody” and the like as used herein refers to whole antibodies that interact with (e.g., by binding, steric hindrance, stabilizing/destabilizing, spatial distribution) a CD40. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelid antibodies, or chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass, preferably IgG and most preferably IgG1. Exemplary antibodies include CFZ533 (herein also designated mAb1) and mAb2, as set forth in Table 1.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen binding site or amino-terminus of the antibody. The N-terminus is a variable region and at the C-terminus is a constant region; the CH3 and CL domains actually comprise the carboxy-terminus of the heavy and light chain, respectively. In particular, the term “antibody” specifically includes an IgG-scFv format.

The “Complementarity Determining Regions” (“CDRs”) are amino acid sequences with boundaries determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). Under IMGT, the CDR regions of an antibody can be determined using the program IMGT/DomainGap Align.

The term “Fc region” as used herein refers to a polypeptide comprising the CH3, CH2 and at least a portion of the hinge region of a constant domain of an antibody. Optionally, an Fc region may include a CH4 domain, present in some antibody classes. An Fc region, may comprise the entire hinge region of a constant domain of an antibody. In one embodiment, the invention comprises an Fc region and a CH1 region of an antibody. In one embodiment, the invention comprises an Fc region CH3 region of an antibody. In another embodiment, the invention comprises an Fc region, a CH1 region and a C_{kappa/lambda} region from the constant domain of an antibody. In one embodiment, a binding molecule of the invention comprises a constant region, e.g., a heavy chain constant region. In one embodiment, such a constant region is modified compared to a wild-type constant region. That is, the polypeptides of the invention disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc.

As used herein, the term “Affinity” refers to the strength of interaction between antibody and antigen at single antigenic sites. Within each antigenic site, the variable region of the antibody “arm” interacts through weak non-covalent forces with the antigen at numerous sites; the more interactions, the stronger the affinity. As used herein, the term “high affinity” for an IgG antibody or fragment thereof (e.g., a Fab fragment) refers to an antibody having a K_{D} of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can vary for other antibody isotypes. For example, high affinity binding for an IgM isotype refers to an antibody having a K_{D} of 10⁻⁷ M or less, or 10⁻⁸ M or less.

As used herein, an antibody or a protein that “specifically binds to CD40 polypeptide” is intended to refer to an antibody or protein that binds to human CD40 polypeptide with a K_{D} of 100 nM or less, 10 nM or less, 1 nM or less.

The term “K_{D}”, as used herein, is intended to refer to the dissociation constant, which is obtained from the ratio of K_{d} to Kₐ (i.e. K_{d}/Kₐ) and is expressed as a molar concentration (M). K_{D} values for antibodies can be determined using methods well established in the art.

A method for determining the K_{D} of an antibody is by using surface plasmon resonance, or using a biosensor system such as a Biacore® system.

As used herein, the term “ADCC” or “antibody-dependent cellular cytotoxicity” activity refers to cell depleting activity. ADCC activity can be measured by the ADCC assay as well known to a person skilled in the art.

As used herein, the term “silent” antibody refers to an antibody that exhibits no or low ADCC activity as measured in an ADCC assay.

In one embodiment, the term “no or low ADCC activity” means that the silent antibody exhibits an ADCC activity that is below 50% specific cell lysis, for example below 10% specific cell lysis as measured in a standard ADCC assay. No ADCC activity means that the silent antibody exhibits an ADCC activity (specific cell lysis) that is below 1%.

Silenced ADCC effector functions can be obtained by mutation in the Fc region of the antibodies and have been described in the art: LALA and N297A (Strohl, W., 2009, Curr. Opin. Biotechnol. vol. 20(6):685-691); and D265A (Baudino et al., 2008, J. Immunol. 181:6664-69; Strohl, W., supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the N297A mutation, which results in aglycosylated/non-glycosylated antibodies.

The term “treatment” or “treat” is herein defined as the application or administration of an anti-CD40 antibody or antigen binding fragment thereof according to the disclosure, for example, mAb1 or mAb2 antibody, to a subject, or application or administration a pharmaceutical composition comprising said anti-CD40 antibody or antigen binding fragment thereof of the invention to an isolated tissue or cell line from a subject, where the subject has an autoimmune disease and/or inflammatory disease, a symptom associated with an autoimmune disease and/or inflammatory disease, or a predisposition toward development of an autoimmune disease and/or inflammatory disease, where the purpose is to alleviate, ameliorate, or improve the autoimmune disease and/or inflammatory disease, any associated symptoms of the autoimmune disease and/or inflammatory disease, or the predisposition toward the development of the autoimmune disease and/or inflammatory disease, in a particular in patients having developed T1DM or suffering from insulits.

By “treatment” is also intended the application or administration of a pharmaceutical composition comprising an anti-CD40 antibodies or antigen binding fragment thereof of the invention, for example, mAb1 or mAb2 antibody, to a subject, or application or administration of a pharmaceutical composition comprising said anti-CD40 antibody or antigen binding fragment thereof of the invention to an isolated tissue or cell line from a subject, where the subject has an autoimmune disease and/or inflammatory disease, a symptom associated with an autoimmune disease and/or inflammatory disease, or a predisposition toward development of an autoimmune disease and/or inflammatory disease, where the purpose is to alleviate, ameliorate, or improve the autoimmune disease and/or inflammatory disease, any associated symptoms of the autoimmune disease and/or inflammatory disease, or the predisposition toward the development of the autoimmune disease and/or inflammatory disease.

The term “prevent” or “preventing” refer to prophylactic or preventative treatment; it is concerned about delaying the onset of, or preventing the onset of the disease, disorders and/or symptoms associated thereto.

As used herein, a subject is “in need of” a treatment if such subject would benefit biologically, medically or in quality of life from such treatment.

The term “pharmaceutically acceptable” means a nontoxic material that does not interfere with the effectiveness of the biological activity of the active ingredient(s).

As used herein, the term “administration” or “administering” of the subject compound means providing a compound of the invention and prodrugs thereof to a subject in need of treatment. Administration “in combination with” one or more further therapeutic agents includes simultaneous (concurrent) and consecutive administration in any order, and in any route of administration. One administration may be a single injection, or multiple injections delivered in conjunction with each other, depending on how much drug substance needs to be administered to achieve therapeutic effect.

As used herein, a “therapeutically effective amount” refers to an amount of an anti-CD40 antibody or antigen binding fragment thereof, e.g., mAb1, that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., an anti-CD40 antibody, e.g., mAb1) administered alone, the term refers to that ingredient alone. When applied to a combination, the term refers to combined amounts of the active ingredients that result in the therapeutic effect, whether administered in combination, serially or simultaneously.

The phrase “therapeutic regimen” means the regimen used to treat an illness, e.g., the dosing protocol used during the treatment of T1DM. A therapeutic regimen may include a loading regimen (or loading dosing), followed by a maintenance regimen (or maintenance dosing).

The phrase “loading regimen” or “loading period” refers to a treatment regimen (or the portion of a treatment regimen) that is used for the initial treatment of a disease. In some embodiments, the disclosed methods, uses, kits, processes and regimens (e.g., methods of treating T1DM) employ a loading regimen (or loading dosing). In some cases, the loading period is the period until maximum efficacy is reached. The general goal of a loading regimen is to provide a high level of drug to a patient during the initial period of a treatment regimen. A loading regimen may include administering a greater dose of the drug than a physician would employ during maintenance regimen, administering a drug more frequently than a physician would administer the drug during a maintenance regimen, or both. Dose escalation may occur during or after the loading regimen.

The phrase “maintenance regimen” or “maintenance period” refers to a treatment regimen (or the portion of a treatment regimen) that is used for the maintenance of a patient during treatment of an illness, e.g., to keep the patient in remission for long periods of time (months or years) following the loading regimen or period. In some embodiments, the disclosed methods, uses and regimens employ a maintenance regimen. A maintenance regimen may employ continuous therapy (e.g., administering a drug at a regular intervals, e.g., weekly, bi-weekly or monthly (every 4 weeks), yearly, etc.) or intermittent therapy (e.g., interrupted treatment, intermittent treatment, treatment at relapse, or treatment upon achievement of a particular predetermined criteria [e.g., pain, disease manifestation, etc.]). Dose escalation may occur during a maintenance regimen.

The phrase “means for administering” is used to indicate any available implement for systemically administering a drug to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

As used herein, the phrase “container having a sufficient amount of the anti-CD40 antibody to allow delivery of [a designated dose]” is used to mean that a given container (e.g., vial, pen, syringe) has disposed therein a volume of an anti-CD40 antibody (e.g., as part of a pharmaceutical composition) that can be used to provide a desired dose. As an example, if a desired dose is 500 mg, then a clinician may use 2 ml from a container that contains an anti-CD40 antibody formulation with a concentration of 250 mg/ml, 1 ml from a container that contains an anti-CD40 antibody formulation with a concentration of 500 mg/ml, 0.5 ml from a container contains an anti-CD40 antibody formulation with a concentration of 1000 mg/ml, etc. In each such case, these containers have a sufficient amount of the anti-CD40 antibody to allow delivery of the desired 500 mg dose.

As used herein, the phrase “formulated at a dosage to allow [route of administration] delivery of [a designated dose]” is used to mean that a given pharmaceutical composition can be used to provide a desired dose of an anti-CD40 antibody, e.g., mAb1, via a designated route of administration (e.g., s.c. or i.v.). As an example, if a desired subcutaneous dose is 500 mg, then a clinician may use 2 ml of an anti-CD40 antibody formulation having a concentration of 250 mg/ml, 1 ml of an anti-CD40 antibody formulation having a concentration of 500 mg/ml, 0.5 ml of an anti-CD40 antibody formulation having a concentration of 1000 mg/ml, etc. In each such case, these anti-CD40 antibody formulations are at a concentration high enough to allow subcutaneous delivery of the anti-CD40 antibody. Subcutaneous delivery typically requires delivery of volumes of less than about 2 ml, preferably a volume of about 1 ml or less. However, higher volumes may be delivered over time using, e.g, a patch/pump mechanism.

The phrase “means for administering” is used to indicate any available implement for systemically administering a drug top a patient, including, but not limited to, a pre-filled syringe, a vial and syringe, an injection pen, an autoinjector, an i.v. drip and bag, a pump, patch/pump, etc. With such items, a patient may self-administer the drug (i.e., administer the drug on their own behalf) or a care-giver or a physician may administer the drug.

### Anti-CD40 Antibodies

Anti-CD40 mAbs with silenced ADCC activity have been disclosed in U.S. Pat. Nos. 8,828,396 and 9,221,913. Anti-CD40 mAbs with silenced ADCC activity are predicted to have an improved safety profile relative to other anti-CD40 antibodies, and in particular may be more suitable for non-oncologic indications, such as T1DM and/or insulitis.

According to a non-binding hypothesis of the inventors, the two mAbs from U.S. Pat. Nos. 8,828,396 and 9,221,913, designated mAb1 and mAb2, are thought to be suitable compounds for treatment of T1DM and/or insulitis. The antibody mAb1, also called CFZ533, is particularly preferred. CFZ533 (iscalimab) is a fully human, immunoglobulin G1 (IgG1) anti-CD40 antibody that blocks recombinant(r) CD154-induced CD40 signaling and which does not cause depletion of CD40 expressing cell types (i.e. Fc-silent; silenced ADCC activity).

mAb1 inhibits CD154-induced activation in vitro and T cell-dependent antibody formation and germinal center formation in vivo. In patients with T1DM or insulitis, CD40 blockade with mAb1 is envisaged to offer a new treatment modality (Example 7).

To enable a person skilled in the art to practice the invention, the amino acid and nucleotide sequences of mAb1 and mAb2 are provided in Table 1 below.

Another anti-CD40 mAb known in the art is ASKP1240 from Astellas Pharma/Kyowa Hakko Kirin Co, as described e.g. in U.S. Pat. No. 8,568,725B2.

Yet another anti-CD40 mAb known in the art is BI655064 from Boehringer Ingelheim, as described e.g. in U.S. Pat. No. 8,591,900.

A further anti-CD40 mAb known in the art is FFP104 by Fast Forward Pharmaceuticals, as described e.g. in U.S. Pat. No. 8,669,352.

TABLE 1
Sequence table

| SEQ | | |
|---|---|---|
| ID | Description | Detailed amino acid |
| NO: | of sequence | or nucleotide sequences |
| 1 | HCDR1 of mAb 1 | SYGMH |
| | and mAb2 (Kabat) | |
| 2 | HCDR2 of mAb 1 | VISYEESNRYHADSVKG |
| | and mAb2 (Kabat) | |
| 3 | HCDR3 of mAb 1 | DGGIAAPGPDY |
| | and mAb2 (Kabat) | |
| 4 | LCDR1 of mAb 1 | RSSQSLLYSNGYNYLD |
| | and mAb2 (Kabat) | |
| 5 | LCDR2 of mAb 1 | LGSNRAS |
| | and mAb2 (Kabat) | |
| 6 | LCDR3 of mAb 1 | MQARQTPFT |
| | and mAb2 (Kabat) | |
| 7 | Variable Heavy | QVQLVESGGGVVQPGRSLRLSCAASGFTFSSYGM |
| | chain of mAb1 and | HWVRQAPGKGLEWVAVISYEESNRYHADSVKGR |
| | mAb2 | FTISRDNSKITLYLQMNSLRTEDTAVYYCARDGGI |
| | | AAPGPDYWGQGTLVTVSS |
| 8 | Variable light chain | DIVMTQSPLSLTVTPGEPASISCRSSQSLLYSNGYN |
| | of mAb1 and mAb2 | YLDWYLQKPGQSPQVLISLGSNRASGVPDRFSGS |
| | | GSGTDFTLKISRVEAEDVGVYYCMQARQTPFTFG |
| | | PGTKVDIR |
| 9 | Full length heavy | QVQLVESGGGVVQPGRSLRLSCAASGFTFSSYGM |
| | chain of mAb1 | HWVRQAPGKGLEWVAVISYEESNRYHADSVKGR |
| | | FTISRDNSKITLYLQMNSLRTEDTAVYYCARDGGI |
| | | AAPGPDYWGQGTLVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVNH |
| | | KPSNTKVDKRVEPKSCDKTHTCPPCPAPELLGGPS |
| | | VFLFPPKPKDTLMISRTPEVTCVVVDVSHEDPEVK |
| | | FNWYVDGVEVHNAKTKPREEQYASTYRVVSVLT |
| | | VLHQDWLNGKEYKCKVSNKALPAPIEKTISKAKG |
| | | QPREPQVYTLPPSREEMTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 10 | Full length light | DIVMTQSPLSLTVTPGEPASISCRSSQSLLYSNGYN |
| | chain of mAb1 | YLDWYLQKPGQSPQVLISLGSNRASGVPDRFSGS |
| | | GSGTDFTLKISRVEAEDVGVYYCMQARQTPFTFG |
| | | PGTKVDIRRTVAAPSVFIFPPSDEQLKSGTASVVCL |
| | | LNNFYPREAKVQWKVDNALQSGNSQESVTEQDS |
| | | KDSTYSLSSTLTLSKADYEKHKVYACEVTHQGLS |
| | | SPVTKSFNRGEC |
| 11 | Full length heavy | QVQLVESGGGVVQPGRSLRLSCAASGFTFSSYGM |
| | chain of mAb2 | HWVRQAPGKGLEWVAVISYEESNRYHADSVKGR |
| | | FTISRDNSKITLYLQMNSLRTEDTAVYYCARDGGI |
| | | AAPGPDYWGQGTLVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNVNH |
| | | KPSNTKVDKRVEPKSCDKTHTCPPCPAPELLGGPS |
| | | VFLFPPKPKDTLMISRTPEVTCVVVAVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSREEMTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 12 | Full length light | DIVMTQSPLSLTVTPGEPASISCRSSQSLLYSNGYN |
| | chain of mAb2 | YLDWYLQKPGQSPQVLISLGSNRASGVPDRFSGS |
| | | GSGTDFTLKISRVEAEDVGVYYCMQARQTPFTFG |
| | | PGTKVDIRRTVAAPSVFIFPPSDEQLKSGTASVVCL |
| | | LNNFYPREAKVQWKVDNALQSGNSQESVTEQDS |
| | | KDSTYSLSSTLTLSKADYEKHKVYACEVTHQGLS |
| | | SPVTKSFNRGEC |
| 13 | Fc region of mAb1 | APELLGGPSVFLFPPKPKDTLMISRTPEVTCVVVD |
| | | VSHEDPEVKFNWYVDGVEVHNAKTKPREEQYAS |
| | | TYRVVSVLTVLHQDWLNGKEYKCKVSNKALPAPI |
| | | EKTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK |
| 14 | Fc region of mAb2 | APELLGGPSVFLEPPKPKDTLMISRTPEVTCVVVA |
| | | VSHEDPEVKFNWYVDGVEVHNAKTKPREEQYNS |
| | | TYRVVSVLTVLHQDWLNGKEYKCKVSNKALPAPI |
| | | EKTISKAKGQPREPQVYTLPPSREEMTKNQVSLTC |
| | | LVKGFYPSDIAVEWESNGQPENNYKTTPPVLDSD |
| | | GSFFLYSKLTVDKSRWQQGNVFSCSVMHEALHN |
| | | HYTQKSLSLSPGK |
| 15 | DNA encoding Full | CAGGTGCAGCTGGTGGAATCTGGCGGCGGAGTG |
| | length heavy chain | GTGCAGCCTGGCCGGTCCCTGAGACTGTCTTGC |
| | of mAb1 | GCCGCCTCCGGCTTCACCTTCTCCAGCTACGGC |
| | | ATGCACTGGGTGCGACAGGCCCCTGGCAAGGG |
| | | ACTGGAATGGGTGGCCGTGATCTCCTACGAGGA |
| | | ATCCAACAGATACCACGCTGACTCCGTGAAGGG |
| | | CCGGTTCACAATCTCCCGGGACAACTCCAAGAT |
| | | CACCCTGTACCTGCAGATGAACTCCCTGCGGAC |
| | | CGAGGACACCGCCGTGTACTACTGCGCCAGGGA |
| | | CGGAGGAATCGCCGCTCCTGGACCTGATTATTG |
| | | GGGCCAGGGCACCCTGGTGACAGTGTCCTCCGC |
| | | TAGCACCAAGGGCCCCTCCGTGTTCCCTCTGGC |
| | | CCCCTCCAGCAAGTCCACCTCTGGCGGCACCGC |
| | | CGCTCTGGGCTGCCTGGTGAAAGACTACTTCCC |
| | | CGAGCCCGTGACCGTGTCCTGGAACTCTGGCGC |
| | | CCTGACCTCCGGCGTGCACACCTTTCCAGCCGT |
| | | GCTGCAGTCCTCCGGCCTGTACTCCCTGTCCTCC |
| | | GTGGTGACCGTGCCCTCTAGCTCTCTGGGCACC |
| | | CAGACCTACATCTGCAACGTGAACCACAAGCCC |
| | | TCCAACACCAAGGTGGACAAGCGGGTGGAACC |
| | | CAAGTCCTGCGACAAGACCCACACCTGTCCCCC |
| | | CTGCCCTGCCCCTGAACTGCTGGGCGGACCTTC |
| | | CGTGTTCCTGTTCCCCCCAAAGCCCAAGGACAC |
| | | CCTGATGATCTCCCGGACCCCCGAAGTGACCTG |
| | | CGTGGTGGTGGACGTGTCCCACGAGGACCCTGA |
| | | AGTGAAGTTCAATTGGTACGTGGACGGCGTGGA |
| | | AGTGCACAACGCCAAGACCAAGCCCAGAGAGG |
| | | AACAGTACGCCTCCACCTACCGGGTGGTGTCTG |
| | | TGCTGACCGTGCTGCACCAGGACTGGCTGAACG |
| | | GCAAAGAGTACAAGTGCAAGGTCTCCAACAAG |
| | | GCCCTGCCTGCCCCCATCGAAAAGACCATCTCC |
| | | AAGGCCAAGGGCCAGCCCCGCGAGCCACAGGT |
| | | GTACACACTGCCCCCCAGCCGGGAAGAGATGAC |
| | | CAAGAACCAGGTGTCCCTGACCTGTCTGGTCAA |
| | | AGGCTTCTACCCCTCCGATATCGCCGTGGAGTG |
| | | GGAGTCCAACGGACAGCCCGAGAACAACTACA |
| | | AGACCACCCCCCCTGTGCTGGACTCCGACGGCT |
| | | CATTCTTCCTGTACTCCAAGCTGACCGTGGACA |
| | | AGTCCCGGTGGCAGCAGGGCAACGTGTTCTCCT |
| | | GCTCCGTGATGCACGAGGCCCTGCACAACCACT |
| | | ACACCCAGAAGTCCCTGTCCCTGAGCCCCGGCA |
| | | AG |
| 16 | DNA encoding Full | GACATCGTGATGACCCAGTCCCCCCTGTCCCTG |
| | length light chain of | ACCGTGACACCTGGCGAGCCTGCCTCTATCTCC |
| | mAb1 | TGCAGATCCTCCCAGTCCCTGCTGTACTCCAAC |
| | | GGCTACAACTACCTGGACTGGTATCTGCAGAAG |
| | | CCCGGCCAGTCCCCACAGGTGCTGATCTCCCTG |
| | | GGCTCCAACAGAGCCTCTGGCGTGCCCGACCGG |
| | | TTCTCCGGCTCTGGCTCTGGCACCGACTTCACAC |
| | | TGAAGATCTCACGGGTGGAAGCCGAGGACGTG |
| | | GGCGTGTACTACTGCATGCAGGCCCGGCAGACC |
| | | CCCTTCACCTTCGGCCCTGGCACCAAGGTGGAC |
| | | ATCCGGCGTACGGTGGCCGCTCCCAGCGTGTTC |
| | | ATCTTCCCCCCCAGCGACGAGCAGCTGAAGAGC |
| | | GGCACCGCCAGCGTGGTGTGCCTGCTGAACAAC |
| | | TTCTACCCCCGGGAGGCCAAGGTGCAGTGGAAG |
| | | GTGGACAACGCCCTGCAGAGCGGCAACAGCCA |
| | | GGAGAGCGTCACCGAGCAGGACAGCAAGGACT |
| | | CCACCTACAGCCTGAGCAGCACCCTGACCCTGA |
| | | GCAAGGCCGACTACGAGAAGCATAAGGTGTAC |
| | | GCCTGCGAGGTGACCCACCAGGGCCTGTCCAGC |
| | | CCCGTGACCAAGAGCTTCAACAGGGGCGAGTGC |
| 17 | DNA encoding Full | CAGGTGCAGCTGGTGGAATCTGGCGGCGGAGTG |
| | length heavy chain | GTGCAGCCTGGCCGGTCCCTGAGACTGTCTTGC |
| | of mAb2 | GCCGCCTCCGGCTTCACCTTCTCCAGCTACGGC |
| | | ATGCACTGGGTGCGACAGGCCCCTGGCAAGGG |
| | | ACTGGAATGGGTGGCCGTGATCTCCTACGAGGA |
| | | ATCCAACAGATACCACGCTGACTCCGTGAAGGG |
| | | CCGGTTCACAATCTCCCGGGACAACTCCAAGAT |
| | | CACCCTGTACCTGCAGATGAACTCCCTGCGGAC |
| | | CGAGGACACCGCCGTGTACTACTGCGCCAGGGA |
| | | CGGAGGAATCGCCGCTCCTGGACCTGATTATTG |
| | | GGGCCAGGGCACCCTGGTGACAGTGTCCTCCGC |
| | | TAGCACCAAGGGCCCCTCCGTGTTCCCTCTGGC |
| | | CCCCTCCAGCAAGTCCACCTCTGGCGGCACCGC |
| | | CGCTCTGGGCTGCCTGGTGAAAGACTACTTCCC |
| | | CGAGCCCGTGACCGTGTCCTGGAACTCTGGCGC |
| | | CCTGACCTCCGGCGTGCACACCTTTCCAGCCGT |
| | | GCTGCAGTCCTCCGGCCTGTACTCCCTGTCCTCC |
| | | GTGGTGACCGTGCCCTCTAGCTCTCTGGGCACC |
| | | CAGACCTACATCTGCAACGTGAACCACAAGCCC |
| | | TCCAACACCAAGGTGGACAAGCGGGTGGAACC |
| | | CAAGTCCTGCGACAAGACCCACACCTGTCCCCC |
| | | CTGCCCTGCCCCTGAACTGCTGGGCGGACCTTC |
| | | CGTGTTCCTGTTCCCCCCAAAGCCCAAGGACAC |
| | | CCTGATGATCTCCCGGACCCCCGAAGTGACCTG |
| | | CGTGGTGGTGGCCGTGTCCCACGAGGACCCTGA |
| | | AGTGAAGTTCAATTGGTACGTGGACGGCGTGGA |
| | | AGTGCACAACGCCAAGACCAAGCCCAGAGAGG |
| | | AACAGTACAACTCCACCTACCGGGTGGTGTCTG |
| | | TGCTGACCGTGCTGCACCAGGACTGGCTGAACG |
| | | GCAAAGAGTACAAGTGCAAGGTCTCCAACAAG |
| | | GCCCTGCCTGCCCCCATCGAAAAGACCATCTCC |
| | | AAGGCCAAGGGCCAGCCCCGCGAGCCACAGGT |
| | | GTACACACTGCCCCCCAGCCGGGAAGAGATGAC |
| | | CAAGAACCAGGTGTCCCTGACCTGTCTGGTCAA |
| | | AGGCTTCTACCCCTCCGATATCGCCGTGGAGTG |
| | | GGAGTCCAACGGACAGCCCGAGAACAACTACA |
| | | AGACCACCCCCCCTGTGCTGGACTCCGACGGCT |
| | | CATTCTTCCTGTACTCCAAGCTGACCGTGGACA |
| | | AGTCCCGGTGGCAGCAGGGCAACGTGTTCTCCT |
| | | GCTCCGTGATGCACGAGGCCCTGCACAACCACT |
| | | ACACCCAGAAGTCCCTGTCCCTGAGCCCCGGCA |
| | | AG |
| 18 | DNA encoding Full | GACATCGTGATGACCCAGTCCCCCCTGTCCCTG |
| | length light chain of | ACCGTGACACCTGGCGAGCCTGCCTCTATCTCC |
| | mAb2 | TGCAGATCCTCCCAGTCCCTGCTGTACTCCAAC |
| | | GGCTACAACTACCTGGACTGGTATCTGCAGAAG |
| | | CCCGGCCAGTCCCCACAGGTGCTGATCTCCCTG |
| | | GGCTCCAACAGAGCCTCTGGCGTGCCCGACCGG |
| | | TTCTCCGGCTCTGGCTCTGGCACCGACTTCACAC |
| | | TGAAGATCTCACGGGTGGAAGCCGAGGACGTG |
| | | GGCGTGTACTACTGCATGCAGGCCCGGCAGACC |
| | | CCCTTCACCTTCGGCCCTGGCACCAAGGTGGAC |
| | | ATCCGGCGTACGGTGGCCGCTCCCAGCGTGTTC |
| | | ATCTTCCCCCCCAGCGACGAGCAGCTGAAGAGC |
| | | GGCACCGCCAGCGTGGTGTGCCTGCTGAACAAC |
| | | TTCTACCCCCGGGAGGCCAAGGTGCAGTGGAAG |
| | | GTGGACAACGCCCTGCAGAGCGGCAACAGCCA |
| | | GGAGAGCGTCACCGAGCAGGACAGCAAGGACT |
| | | CCACCTACAGCCTGAGCAGCACCCTGACCCTGA |
| | | GCAAGGCCGACTACGAGAAGCATAAGGTGTAC |
| | | GCCTGCGAGGTGACCCACCAGGGCCTGTCCAGC |
| | | CCCGTGACCAAGAGCTTCAACAGGGGCGAGTGC |
| 19 | Amino acid | MVRLPLQCVLWGCLLTAVHPEPPTACREKQYLIN |
| | sequence of human | SQCCSLCQPGQKLVSDCTEFTETECLPCGESEFLD |
| | CD40 | TWNRETHCHQHKYCDPNLGLRVQQKGTSETDTIC |
| | | TCEEGWHCTSEACESCVLHRSCSPGFGVKQIATG |
| | | VSDTICEPCPVGFFSNVSSAFEKCHPWTSCETKDL |
| | | VVQQAGTNKTDVVCGPQDRLRALVVIPIIFGILFAI |
| | | LLVLVFIKKVAKKPTNKAPHPKQEPQEINFPDDLP |
| | | GSNTAAPVQETLHGCQPVTQEDGKESRISVQERQ |

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. The following Examples are presented in order to more fully illustrate the preferred embodiments of the disclosure. These examples should in no way be construed as limiting the scope of the disclosed patient matter, as defined by the appended claims.

### EXAMPLES

### General Methodology

### Example 1: Expression Systems

For expression of the light and heavy chains, the expression vector(s) encoding the heavy and light chains are transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the invention in either prokaryotic or eukaryotic host cells. Expression of antibodies in eukaryotic cells, for example mammalian host cells, yeast or filamentous fungi, is discussed because such eukaryotic cells, and in particular mammalian cells, are more likely than prokaryotic cells to assemble and secrete a properly folded and immunologically active antibody.

Particularly a cloning or expression vector can comprise either at least one of the following coding sequences (a)-(b), operatively linked to suitable promoter sequences:

(a) SEQ ID NO: 15 and SEQ ID NO: 16 encoding respectively the full length heavy and light chains of mAb1; or

(b) SEQ ID NO: 17 and SEQ ID NO: 18 encoding respectively the full length heavy and light chains of mAb2.

Mammalian host cells for expressing the recombinant antibodies of the invention include Chinese Hamster Ovary (CHO cells) (including dhfr-CHO cells, described Urlaub and Chasin, 1980 Proc. Natl. Acad. Sci. USA 77:4216-4220 used with a DH FR selectable marker, e.g., as described in R. J. Kaufman and P. A. Sharp, 1982 Mol. Biol. 159:601-621), CHOK1 dhfr+ cell lines, NSO myeloma cells, COS cells and SP2 cells. In particular, for use with NSO myeloma cells, another expression system is the GS gene expression system shown in PCT Publications WO 87/04462, WO 89/01036 and EP0338841.

When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods (See for example Abhinav et al. 2007, Journal of Chromatography 848: 28-37).

The host cells may be cultured under suitable conditions for the expression and production of mAb1 or mAb2.

### Example 2. Pharmacology

### 1. Primary Pharmacology

mAb1 binds to human CD40 with high affinity (K_{d} of 0.3 nM). However, it does not bind to Fcγ receptors (including CD16) or mediate antibody-dependent cellular cytotoxicity or complement-dependent cytotoxicity. mAb1 inhibits recombinant CD154 (rCD154)-induced activation of human leukocytes, but does not induce PBMC proliferation or cytokine production by monocyte-derived dendritic cells (DCs). mAb1 binds human and non-human primate CD40 with very similar affinities.

In vivo, mAb1 blocks primary and secondary T cell-dependent antibody responses (TDAR), and can prolong survival of kidney allografts in non-human primates (Cordoba et al 2015). In addition, mAb1 can disrupt established germinal centers (GCs) in vivo.

The CD40 receptor occupancy and functional activity were simultaneously assessed in vitro using human whole blood cultures. Functional activity was quantified via CD154-induced expression of CD69 (the activation marker) on CD20 positive cells (B cells) and CD40 occupancy was monitored using fluorescently labeled mAb1. Almost complete CD40 occupancy by mAb1 was required for full inhibition of rCD154-induced CD69 expression.

2. Secondary Pharmacology

The effects of mAb1 on platelet function and blood hemostasis were investigated, indicating that mAb1 does not induce platelet aggregation responses, rather displays certain mild inhibitory effects on platelet aggregation at high concentrations.

### Example 3. Non-Clinical Toxicology and Safety Pharmacology

Toxicology studies with mAb1 did not reveal any significant organ toxicities, including no evidence of thromboembolic events as reported in clinical trials with anti-CD154 mAbs (Kawai et al 2000). In a 26-week chronic toxicity study in cynomolgus monkeys, no adverse, mAb1-related findings were discovered. Based on these data, the NOAEL was set at 150 mg/kg (26-week). The mean (all animals) C_{max,ss} was 44, 3235, and 9690 μg/mL at 1, 50, and 150 (NOAEL) mg/kg S.C. weekly, respectively. The NOAEL derived from the 26-week cynomolgus monkey study is considered the most relevant for supporting the clinical dosing regimen.

Because of the complete inhibition of T cell-dependent antibody responses (TDAR), KLH, the formation of anti-drug antibodies (ADA) to mAb1 is not expected and therefore ADA-related side effects are considered unlikely when concentrations of mAb1 are maintained continuously at pharmacological levels.

Tissue cross-reactivity studies revealed that CD40 is not only present on immune cells, but also in various tissues. This is mainly due to its expression on endothelial and epithelial cells, where CD40 is involved in signaling such as responding to wound healing processes, upregulation of virus-defense, and inflammatory-related mediators. An antagonistic anti-CD40 monoclonal antibody like mAb1 is not expected to contribute to inflammatory processes, which was confirmed by in vitro studies using human umbilical vein endothelial cells (HUVEC).

In conclusion, the nonclinical data support the a study using an anti-CD40 antibody with silenced ADCC activity in patients with primary T1DM.

### Example 4: Study Design

This is Investigator- and subject-blinded, randomized, placebo-controlled study to evaluate safety, tolerability, pharmacokinetics and efficacy of CFZ533 in pediatric and young adults with new onset type 1 diabetes mellitus (T1DM)

1. Purpose and Rationale

The purpose of this study is to provide clinical data to enable the development of CFZ533 in new onset T1DM.

2. Primary Objective(s)

To evaluate safety and tolerability of CFZ533 in new onset T1DM by assessing adverse events (AEs) and standard safety labs.

To evaluate the treatment effect of CFZ533 on pancreatic beta cell function in new onset T1DM after 1 year, by assessing stimulated C-peptide AUC by mixed meal tolerance test (MMTT).

The mixed meal tolerance test (MMTT) has appropriate sensitivity to detect residual insulin secretion and beta cell function. In the MMTT, a weight-based liquid meal provided as 6 mL/kg (maximum 360 mL) of mixed meal, ingested over 5 min with timed blood samples for glucose and C-peptide determination obtained 10 min prior to ingestion (t=−10), at baseline (t=0), and at 15, 30, 60, 90, and 120 min after completing consumption of the liquid meal (Leighton et al 2017).

Blood sample collection will allow measurement of area under the curve (AUC0-2 hr) and peak C-peptide values. MMTT will be collected at baseline, at Weeks 12, 24, 36 and 52 on treatment, and Months 18, 24 and 36 post treatment. The primary efficacy endpoint is the treatment effect on stimulated C-peptide AUC0-2 hrs by mixed meal tolerance test (MMTT) at 12 months. Proportion of subjects with detectable C-peptide, rate of decline from baseline C peptide; time to undetectable C peptide will be assessed. A timed urine for C-peptide:creatinine ratio will also be assessed as a measure of beta cell function.

3. Secondary Objective(s)

To evaluate the pharmacokinetics (PK) of CFZ533 in new onset T1DM by measuring free CFZ533 plasma concentrations at baseline, during treatment and follow up period.

To evaluate the treatment effect of CFZ533 on full remission by HbA1c ≤6.5% (48 mmol/mol) and no exogenous insulin use, or partial remission by insulin dose adjusted HbA1c (IDAA1c) ≤9.0 or HbA1c <7.0% (53 mmol/mol) with total daily insulin dose of <0.5 units/kg/day, in new onset T1DM at 1 year.

To evaluate durability of effects of CFZ533 on pancreatic beta cell function in subjects with new-onset T1DM at 2 years after last dose by assessing stimulated C-peptide AUC by MMTT.

TABLE 2
Objectives and related endpoints

| Objective(s) | Endpoint(s) |
|---|---|
| Primary objective(s) | Endpoint(s) for primary objective(s) |
| To evaluate effects of | Stimulated C-peptide AUC by mixed meal |
| CFZ533 on pancreatic | tolerance test (MMTT) after 1 year of |
| beta cell function in | treatment. |
| subjects with new-onset | |
| T1DM. | |
| To evaluate the safety and | Adverse events, safety labs. |
| tolerability of CFZ533 in | |
| subjects with new onset | |
| T1DM. | |
| Secondary objective(s) | Endpoint(s) for secondary objective(s) |
| To evaluate the | CFZ533 plasma concentrations at baseline, |
| pharmacokinetics (PK) of | during treatment (Cmax, Tmax after IV |
| CFZ533 in subjects with | administration, and Ctroughs after SC |
| new onset T1DM. | administration) and follow-up period. |
| To evaluate durability of | Stimulated C-peptide AUC by MMTT at 2 |
| effects of CFZ533 on | years from last dose. |
| pancreatic beta cell function | |
| in subjects with new-onset | |
| T1DM. | |
| To evaluate the treatment | Not requiring exogenous insulin therapy with |
| effect of CFZ533 on | HbA1c < 6.5% (remission) or Insulin dose |
| remission or partial | adjusted HbA1c (IDAA1c ≤ 9.0) or |
| remission in subjects with | HbA1c < 7.0% (53 mmol/mol) and total |
| new onset T1DM. | daily insulin dose < 0.5 units per kg per day |
| | at 1 year (partial remission). |

4. Study Design CCFZ533X2207

CCFZ533X2207 (“study) is a Phase 2, non-confirmatory, investigator- and subject-blinded, randomized, placebo-controlled study to evaluate the safety, tolerability, pharmacokinetics, and efficacy of CFZ533 on preservation of residual pancreatic β-cell function in new onset T1DM in pediatric and young adult subjects.

Newly diagnosed T1DM patients are identified based on American Diabetes Association diagnostic criteria and National Institute for Health and Care Excellence (NICE) (Johnston 2004), and the presence of at least one diabetes-related autoantibody as specified in the section population below. Enrollment in the trial should occur no less than two weeks and within 100 days from the time of diagnosis and first study drug administration. Enrollment will be based on both screening and baseline results. The screening visit may be conducted over 1 or 2 visits depending on the subject's body weight (as described herein).

Approximately 102 subjects aged 6-21 years will be enrolled, to ensure that approximately 81 subjects complete the study. Five sequential study cohorts (based on age and body weight) are planned, but enrollment into previous cohorts will continue until enrollment goals have been met. To allow adequate enrollment into all weight and age cohorts, enrollment into Cohort 1 will be stopped after approximately 50 subjects.

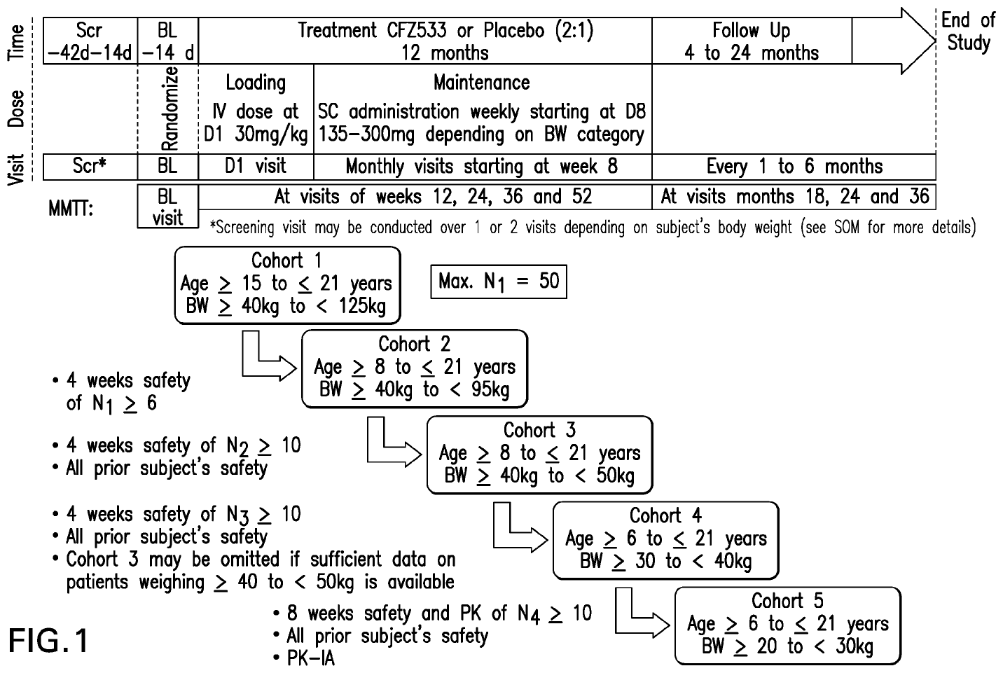

Cohorts are based on descending order of age and weight groups (FIG. 1).

The study is planned for 81 completers at 16 months for primary efficacy (at 12 months) and safety endpoints (at 16 months). Approximately 102 subjects will be enrolled.

Eligible subjects will be enrolled and randomized to active or placebo in a 2:1 ratio.

The study timeline includes a screening period of 6 weeks, a baseline period of 2 weeks, a treatment period of 12 months and a follow-up period after last dose of CFZ533 of 4 months and up to 2 years. Each subject will be in the study for a minimum of approximately 1.5 years, and up to 3 years.

This study will be the first study to evaluate CFZ533 in a pediatric population in any indication. Thus, enrollment will be staggered to evaluate safety and tolerability on a rolling basis and enable gradual enrollment of younger age and lower weight groups.

The PK profile of CFZ533 will be evaluated throughout the study (Month 3, 6 and 9). Dosage adjustments, particularly for Cohort 5 (lowest body weight limit of 20 kg), may be introduced based on emerging PK data in this population (PK Interim Analysis).

Sequential enrollment by age and body weight (BW) categories include:

Cohort 1: age ≥15 to ≤21 years (older adolescents to young adults), BW 40 to 125 kg.

Cohort 2: age ≥8 to ≤21 years (young adolescents to young adults), BW 40 to 95 kg.

Cohort 3 to Cohort 5 differ by age and limit of body weight.

Cohort 3: age ≥8 to ≤21 years, BW ≥40 to <50 kg.

Cohort 4: age ≥6 to ≤21 years, BW ≥30 to <40 kg.

Cohort 5: age ≥6 to ≤21 years, BW ≥20 to <30 kg.

Opening subsequent cohorts (for Cohort 2, Cohort 3, and Cohort 4) will be based on a safety and tolerability review at 4 weeks. Before opening a subsequent cohort, safety and tolerability data from at least 6 subjects in Cohort 1 and 10 subjects in all other cohorts, must be reviewed and deemed safe to proceed by the Sponsor and Lead Investigator. In addition, at least 10 subjects with body weight of ≥40 to <50 kg must be enrolled and evaluated before proceeding to Cohort 4. If 10 subjects in this weight range are evaluable from Cohorts 1 and 2, then Cohort 3 may be omitted. The transition between Cohort 4 and Cohort 5 will occur when at least 10 subjects between 30-40 kg bodyweight complete the first 8 weeks of treatment (including subcutaneous (SC) dosing on Week 8), and will be based on safety and tolerability at 8 weeks and PK data (PK Interim Analysis). Dose adjustment in Cohort 5 may be implemented based on a PK Interim Analysis and safety analysis. Enrollment into previous cohorts will continue until enrollment goals have been met.

After a screening and baseline period of up to 42 days, subjects eligible for a cohort open for enrollment will be randomized to receive either CFZ533 or placebo (2:1 ratio) for one year, in addition to background standard of care with intensive insulin therapy, although the overarching goal is to start treatment as early after diagnosis of T1DM as possible. In the event a subject has not had all vaccinations recommended according to local guidance, the screening period may be extended beyond 42 days to allow these vaccinations to be administered, but first dose of study drug must be administered within 100 days of diagnosis of T1DM.

The treatment includes an initial loading dose of CFZ533 (administered at a dose of 30 mg/kg via a peripheral intravenous (IV) line on Day 1/Week 0; all subjects receive the same dose in mg/kg) or matching placebo. All subsequent doses of CFZ533 (or matching placebo), from Day 8 (Week 1) up to Day 365 (Week 52; last dose) are administered on a weekly basis by subcutaneous (SC) injections at a dose which is based on body weight (see Rationale for dose/regimen and duration of treatment details).

A standardized liquid MMTT to assess β-cell function using C-peptide will be performed at baseline, Week 12, 24, 36, 52, and Month 18, 24 and 36 (or end of study), during the course of the study, as described herein. The following diabetes-related endpoints will also be monitored during the study: HbA1c, urinary C-peptide to creatinine ratio, total daily dose of insulin, serial and continuous glucose measurements, and diabetic ketoacidosis and hypoglycemic events.

In the event the primary analysis, performed when at least 81 randomized and treated subjects complete their 1-year assessment visit, indicates a positive drug effect on beta cell function, all subjects will be followed-up for an interval up to two additional years to evaluate the durability of efficacy on β-cell preservation. In the event that no positive drug effect is seen at the approximately 1-year treatment period for the primary analysis, the follow-up assessment for safety monitoring for each subject will be performed at least 4 months post-last dose of CFZ533 or placebo.

Cohort expansion will be based on the following:
To open Cohort 2: At least 6 subjects (aged 15 to ≤21 years) of Cohort 1 treated with CFZ533 or placebo for 4 weeks along with the safety data for all subjects enrolled up to that point, must be reviewed and deemed satisfactory. Assessment of 6 subjects in this cohort, as opposed to 10 in other cohorts, is because existing safety data is more robust for subjects in this age and weight range population.
To open Cohort 3: At least 10 young adolescent to young adults (aged ≥8 to 21 years) of Cohort 2 treated with CFZ533 or placebo for 4 weeks along with the safety data for all subjects enrolled up to that point, must be reviewed and deemed satisfactory before Cohort 3 (age ≥8 to ≤21 years and body weight (BW) ≥40 to <50 kg) can be opened for enrollment. However, if 10 subjects in this weight range are evaluable from Cohorts 1 and 2, then Cohort 3 may be omitted.
To open Cohort 4: At least 10 children to young adults (aged ≥8 to 21 years, body weight (BW) ≥40 to <50 kg) treated with CFZ533 or placebo for 4 weeks along with the safety data for all subjects enrolled up to that point, must be reviewed and deemed satisfactory before Cohort 4 (age ≥6 to ≤21 years and body weight ≥30 to ≤40 kg) can be opened for enrollment.
To open Cohort 5: PK data from at least 10 children/youth between ≥30 to ≤40 kg BW (Cohort 4) to have completed 8 weeks of treatment, along with the safety data for all subjects enrolled up to that point, will trigger a formal PK Interim Analysis (PK-IA; Section 4.4), and must be reviewed and deemed satisfactory before Cohort 5 (age ≥6 to ≤21 years and body weight 20 to <30 kg) can be opened for enrollment.

Enrollment into previous cohorts will continue throughout the study and will not be stopped when the next cohort opens for enrollment, except for Cohort 1 which will be capped at approximately 50 subjects. A minimum of 10 subjects will be enrolled to each cohort. However, as noted above, if 10 subjects in the ≥40 to <50 kg weight range are evaluable from Cohorts 1 and 2, then Cohort 3 may be omitted.

An interim analysis for futility is planned once at least 50% of enrolled subjects complete 6 months of treatment. An interim analysis for futility and efficacy may be conducted once at least ⅔ of enrolled subjects complete 12 months of treatment.

One year of treatment is required to be able to differentiate potential drug effect on reduction in β-cell function (slowing of the decline) from the natural disease progression, over a clinically relevant duration of time.

5. Population

The study population will be comprised of newly diagnosed T1DM pediatric and young adult subjects. Approximately 102 subjects between the ages of 6 and 21 (inclusive) will be enrolled and randomized in the study in sequential order as outlined in the study design.

6. Key Inclusion Criteria
Newly diagnosed auto immune T1DM confirmed by at least one positive auto antibody: glutamic acid decarboxylase (anti-GAD), protein tyrosine phosphatase-like protein (anti-IA-2); zinc transporter 8 (anti-ZnT8); islet cell (cytoplasmic) (anti-ICA).
Peak stimulated C-peptide levels ≥0.2 pmol/mL (0.6 ng/mL) following standard liquid MMTT within one month prior to randomization.
   Study participants are to complete all recommended inactivated (killed) immunizations at least 4 weeks prior and attenuated (live) immunizations at least 4 months in accordance with local immunization guidelines and prior to first dose with study drug.

7. Key Exclusion Criteria
Diabetes forms other than autoimmune T1DM such as maturity-onset diabetes of the young (MODY), latent autoimmune diabetes of the adult (LADA), acquired diabetes (secondary to medications or surgery), type 2 diabetes.
Diabetic ketoacidosis within 2 weeks of the baseline MMTT test.
History of polyglandular autoimmune disease, Addison's disease, pernicious anemia, celiac sprue.
History of immunodeficiency disorders, such as HyperIgM syndrome; history of recurrent infections suggestive of immune deficiency disorders.
Major dental work within 8 days prior to CFZ533 intravenous loading dose; febrile illness within 48 hours prior to first dose.
Use of other investigational drugs or use of immunosuppressive agents at the time of enrollment, or within 5 half-lives of enrollment, or until the expected pharmacology effect has returned to baseline, whichever is longer; or longer if required by local regulations.
Chronic infection with Hepatitis B (HBV) or Hepatitis C (HCV). A positive HBV surface antigen (HBsAg) test, or if standard local practice, a positive HBV core antigen test, excludes a subject. Subjects with a positive HCV antibody test should have HCV RNA levels measured. Subjects with positive (detectable) HCV RNA should be excluded
Evidence of Epstein-Barr Virus (EBV), Cytomegalovirus (CMV) or Herpes simplex virus (HSV) by viral load above laboratory threshold suggestive of active infection.
Any of the following abnormal laboratory values at screening: total white blood cell count (WBC) outside the range of 1,500-15,000/mm3 (1.5-15.0×109/L).

neutrophil count (<1500/mm3) (<1.5×109/L).

lymphocyte count <500/mm3 (<0.5×109/L).

hemoglobin (Hgb)<8.0 g/dL.
platelets <100,000/mm3 (<100×109/L).

8. Study Treatment

Subjects will be assigned to one of the 2 treatment arms in a ratio of 2:1, CFZ533 or matching placebo.

For the single intravenous (IV) loading dose, all subjects receive the same dose: CFZ533 30 mg/kg IV on Study Day 1 (Week 0).

For the subcutaneous (SC) maintenance regimen, a fixed CFZ533 dose by body weight (BW) category will be administered every week from Day 8 (Week 1) up to Week 52 (last dose):

BW category I (≥20 to <30 kg): 135 mg (1 injection of 0.9 mL) SC weekly.

BW category II (≥30 to <50 kg): 195 mg (1 injection of 1.3 mL) SC weekly.

BW category III (≥50 kg): 300 mg (1 injection of 2 mL, or 2 injections of 1 mL) SC weekly.

9. Efficacy Assessments

C-peptide during MMTT

10. Pharmacodynamic Assessments

Soluble CD40 (sCD40) concentrations in plasma (target biology and target engagement in whole blood) at baseline, during treatment and follow-up period.

11. Pharmacokinetic Assessments

CFZ533 concentrations in plasma (at baseline, during treatment and follow up period). 12. Other assessments

Continuous glucose monitoring.

Potential mode of action, disease and early efficacy biomarkers (including but not limited to Follicular T helper cells, serum CXCL13, a targeted panel of T1DM autoantibodies in serum and urine CD40).

Immunogenicity of CFZ533 (anti-CFZ533 antibodies in plasma at baseline, during treatment and follow-up period).

13. Data Analysis

Primary safety and efficacy analysis will be conducted after at least 81 subjects have completed 12 months of treatment. If there is no evidence of efficacy at 12 months all subjects will be scheduled for an end of study visit when each subject has passed at least 4 months since final dosing to permit post-dosing safety and washout assessments.

For all analyses, subjects will be analyzed according to the study treatment(s) received. The safety analysis set will include all subjects that received any study treatment.

The PK analysis set will include all subjects with at least one available valid CFZ533 concentration measurement, who received any study drug and with no protocol deviations that impact PK data. The PD analysis set will include all subjects with available PD data and without major protocol deviations that affect the PD outcome.

14. Rationale for Dose/Regimen and Duration of Treatment

The dosing rationale for CFZ533 in new onset T1DM subjects is based on exposure, safety and tolerability data from trials with CFZ533 in

Kidney transplant (kidney Tx; Study CCFZ533X2201-Part 2),

Primary Sjögren's Syndrome (pSS; Study CCFZ533X2203),

Graves' disease (GD; Study CCFZ533X2205),

Myasthenia gravis (MG; Study CCFZ533X2204),

Rheumatoid arthritis (RA) subjects (first in human Study CCFZ533X2101), and based on efficacy data in kidney transplant, Primary Sjögren's Syndrome, and Graves' disease.

The dosing regimen includes,

a body weight-adjusted intravenous (IV) loading dose of 30 mg/kg on Day 1 (Week 0) for all subjects in the trial, followed by

a fixed subcutaneous (SC) dose administered weekly from Day 8 (Week 1) to Week 52 (last dose), which is based on the following body weight categories,

body weight category I (≥20 to <30 kg): 135 mg (1 injection of 0.9 mL),

body weight category II (≥30 to <50 kg): 195 mg (1 injection of 1.3 mL),

body weight category III (≥50 kg): 300 mg (1 injection of 2 mL or 2 injections of 1 mL).

The IV loading dose will be administered at the Investigator site. The weekly SC dose (135, 195 or 300 mg) will be defined based on the body weight of the subject recorded every 3 months at site visits on Day 1/Week 0, Day 85/Week 12, Day 169/Week 24, Day 253/Week 36 and Day 337/Week 48 to account for body weight gain or loss during the treatment period. Weekly SC doses may be administered at home or at investigator site.

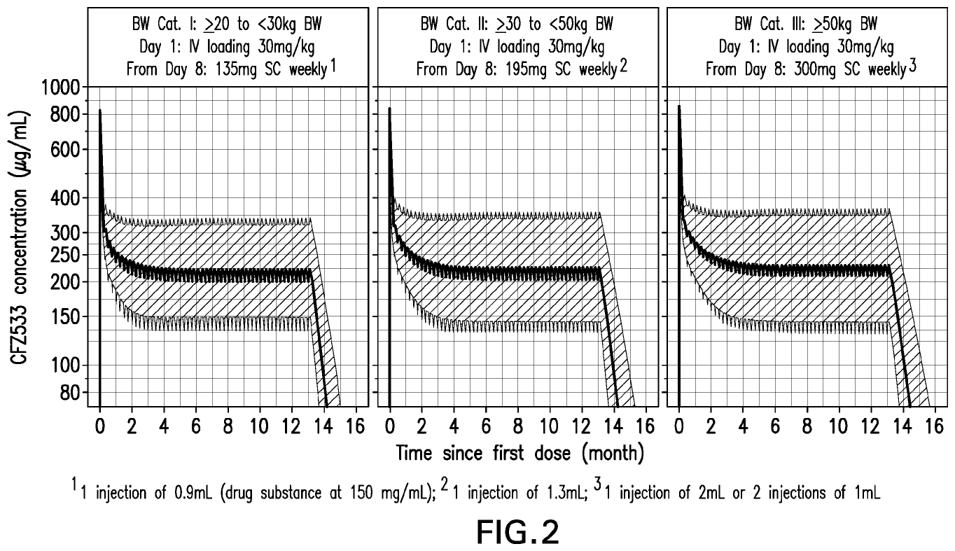

Predicted PK profiles in new onset T1DM subjects by body weight category (similar CFZ533 plasma concentrations are achieved for all three body weight categories) are presented in FIG. 2.

Rationale for the Intravenous (IV) Loading Dose on Day 1

The IV loading dose of 30 mg/kg on Day 1 is predicted:
To rapidly saturate CD40 receptors in target tissues (i.e. pancreatic lymph nodes) and minimize the CD40-mediated elimination of CFZ533 in conditions where the aggressiveness of the disease within 100 days of diagnosis (insulitis, B- and T-lymphocytes infiltration in pancreatic islets, active ectopic germinal centers) is likely to be associated with high tissue CD40 expression,
To rapidly block the aggressive autoimmune destruction of residual p-cells, insulitis and local infiltration of pathogenic auto reactive B lymphocytes.

CD40 expression in new onset T1DM patients:

Patients with autoimmune diseases (including T1DM) generally present with increased CD40 expression in target tissues and elevated serum/plasma soluble CD40 (sCD40; shed receptors) levels.

In T1D patients, elevated plasma sCD40 levels are assumed to reflect elevated expression of CD40 in target tissues.

In Chatzigeorgiou et al (2010a),
Pediatric T1DM patients had significantly higher plasma sCD40 levels (93 μg/mL) compared to healthy controls (66 μg/mL), which were associated with elevated plasma interleukin-6 (IL-6), matrix metalloproteinase-9 (MMP-9) and CRP levels.
Urine sCD40 levels were also elevated in T1DM compared to healthy controls: 335 μg/mL and 150 μg/mL, respectively, suggesting that the elevated plasma levels of sCD40 in these patients reflect increased CD40 production rather than diminished kidney excretion.
Upregulation of cellular CD40 (peripheral blood mononuclear cells) was also observed and was positively correlated with plasma sCD40, IL-6, CRP as well as hemoglobin A1c (HbA1c).
Plasma and peripheral blood mononuclear cell (PBMC) CD40 levels appear to be elevated in pediatric patients with T1DM, and positively correlate with inflammation.

In Chatzigeorgiou et al (2010b), plasma CD40 concentrations were also significantly higher in diabetic patients than in healthy controls (about 110 μg/mL vs. 55 μg/mL) and correlated positively with HbA1c. Moreover, for patients with disease duration <1 month, 1-6 months, or ≥6 months, plasma CD40 was about 75, 190 and 88 μg/mL, respectively.

Achieving saturating and efficacious conditions in pancreatic islets at start of treatment (dosing within 100 days of diagnosis) through an IV loading dose is critical for an effective immune intervention to preserve residual p-cell function.

The CFZ533 30 mg/kg IV loading dose is further justified considering that CFZ533 is subject to CD40-mediated drug disposition (or target mediated drug disposition—TMDD; a process in which a significant proportion of the drug (relative to dose) is bound to CD40 receptors and affects CFZ533 clearance).

The extent of TMDD is dictated by the level of CD40 receptors in tissues and the level of saturation of these receptors. Elevated CD40 expression may be associated with high elimination rate of CFZ533 and loss of target engagement in target tissues if CD40 is not fully saturated.

The loading regimen is expected to provide full CD40-CD154 pathway blockade at start of treatment. In these conditions, the contribution of CD40 to the overall clearance of CFZ533 is minimal, and the disposition of CFZ533 is mainly the consequence of CFZ533 binding to FcRn receptors.

Failure to saturate CD40 receptors in conditions where CD40 expression in tissues is significantly enhanced can translate in clinical efficacy failure.

This was demonstrated with the anti-CD40 antibody ASKP1240 (bleselumab; Goldwater et al 2013).

Bleselumab was investigated in kidney transplant (Tx) subjects (Phase 2 trial; Harland et al 2017) and in transplanted monkeys (Ma et al 2014).

In the Phase 2 trial with bleselumab, most of the rejections in the calcineurin inhibitor-free arm occurred before Day 60. We hypothesize that bleselumab failed in the CNI-free regimen due to under dosing that failed to fully saturate increased levels of CD40 expression in tissues during the first 1 or 2 months, leading to efficient CD40-mediated elimination of bleselumab, sub-optimal tissue exposure and high rates of graft failure.

This hypothesis is supported by preclinical data in transplanted monkeys. When the transplanted monkeys received allograft kidneys, the recipient's immune system was rapidly activated, resulting in an increase in CD40 expression levels in B lymphocytes, dendritic cells, and macrophages as well as on selected allograft parenchymal cells. As a result of the immune response against allo-antigens, the number of activated cells and unoccupied CD40 sites increased and would require higher doses and/or more frequent administration of bleselumab, a notion borne out by an increased clearance of bleselumab observed in transplanted animals.

CFZ533 Exposures after the 30 mg/kg IV Loading Dose

The CFZ533 30 mg/kg IV loading dose is expected to reach exposures (including a median Cmax of about 826 μg/mL) which correspond to doses and regimens which have been used in Rheumatoid arthritis (RA) and kidney transplant patients.
In Rheumatoid arthritis subjects (N=4; first in human study), a mean Cmax of 848 μg/mL (range of 635 to 1120 μg/mL) was observed. This dose was safe and well tolerated
In kidney transplant (N=33 full analysis set; CCFZ533X2201-Part 2), the loading regimen was 10 mg/kg IV on Day 1, 3, 7 (high frequency during the first week), 15, 29, 43 and 57, then, from Day 57 the maintenance regimen was 10 mg/kg IV every 4 weeks. This loading regimen was well tolerated and was not associated with an increased rate of infection for CFZ533 treated subjects compared to controls on background immune suppressive therapy, and with no reports of neutropenia. The highest mean observed trough plasma CFZ533 concentration was 306 μg/mL (range 161-419 μg/mL) on Day 57, at the end of the loading regimen.

In the 13-week toxicology study with CFZ533 (150 mg/kg/week IV) in rhesus monkey, the Cmax (Day 1) and Cmax, at steady state (ss) (Week 13) were 4060 and 11650 μg/mL, respectively. The NOAEL in this study was set at 10 mg/kg due to inflammatory changes in different organs which were considered resulting from a background infection.

There were no other findings in toxicity studies except for immunosuppression-related infections which can be expected based on the pharmacological properties of CFZ533.

Rationale for the subcutaneous (SC) maintenance regimen

As opposed to the body weight (BW)-adjusted approach for the IV loading dose, the weekly SC maintenance regimen which starts on Day 8 up to the last dose on Week 52, is based on a fixed SC dose selected according to the following three body weight categories,
Body weight Category I (20 to <30 kg): 135 mg SC weekly.
Body weight Category II (30 to <50 kg): 195 mg SC weekly.
Body weight Category III (≥50 kg): 300 mg SC weekly.

Assuming a fixed dosing strategy, body weight categories were created to maintain similar exposure levels for all subjects across the body weight range in this study. This is justified based on the anticipated effect of body weight on the clearance of CFZ533. This is typical for a monoclonal Ab like CFZ533 and consistent with allometric principles where a fixed dose strategy is used (Wang et al 2009, Wang and Prueksaritanont 2010).

Three body weight categories are proposed (20 to <30 kg, 30 to <50 kg and ≥50 kg). This is justified to ensure similar between subject variability within each category and based on similar fold-exposure differences between each boundary within a body weight category (1.4 to 1.6-fold assuming an allometric coefficient of 0.75 on body weight, which is also typical for IgG1 type antibodies).

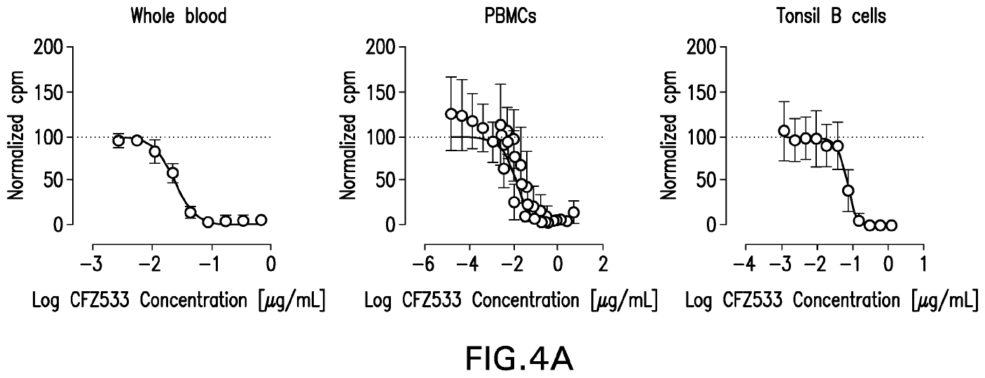

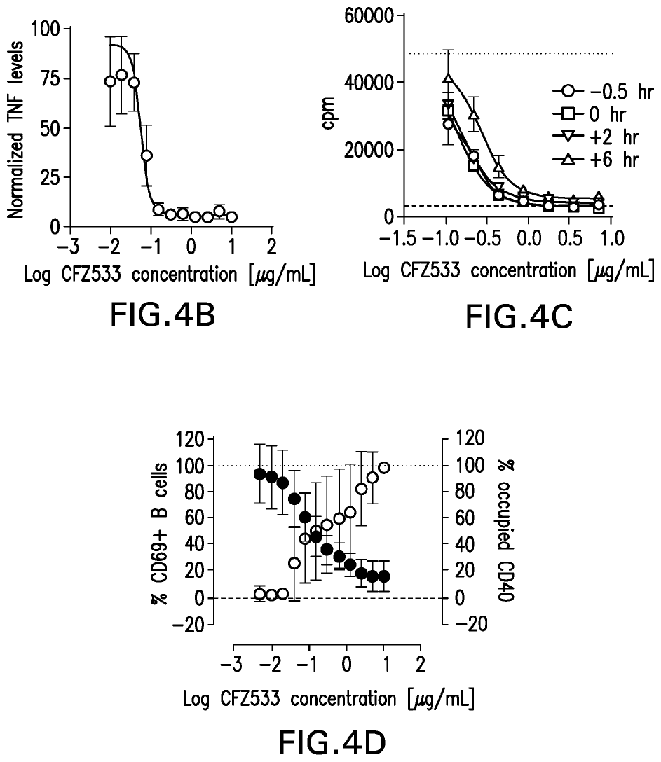

In Category III (≥50 kg body weight), at steady state, the predicted typical trough CFZ533 plasma concentration (Ctrough, ss) is about 222 μg/mL (90% of the population between 140-344 μg/mL; FIG. 4-1). Similar CFZ533 plasma steady state (ss) Ctrough values are predicted for Category I (20 to <30 kg) and Category II (30 to <50 kg).

Predicted CFZ533 plasma Ctrough, ss values in T1DM were already evaluated in ongoing or completed clinical studies with CFZ533 in Primary Sjögren's Syndrome, kidney transplant, Graves' disease and Myasthenia gravis patients, where they were overall safe and well tolerated.

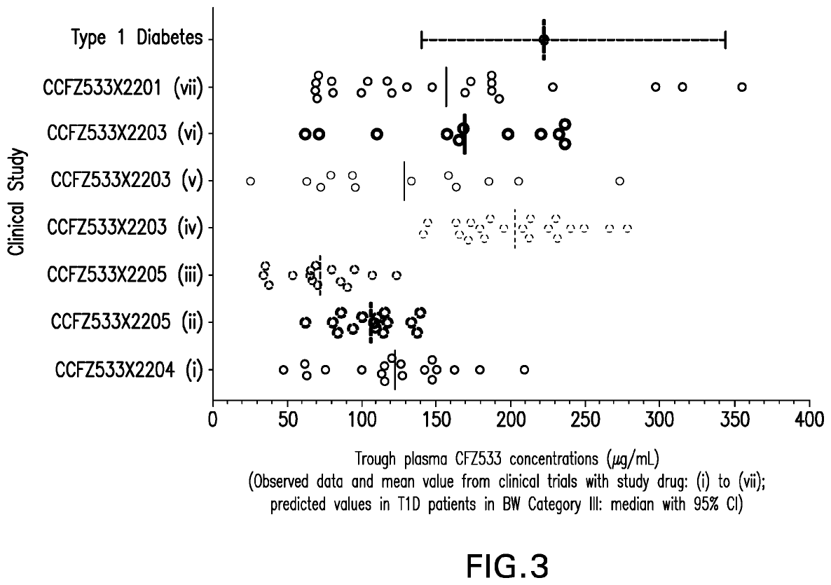

Predicted CFZ533 plasma Ctrough, ss values for T1DM patients/subjects in body weight Category III (similar Ctrough,ss are predicted for Category I and II) are compared to observed trough concentrations for CFZ533 in previous clinical trials in FIG. 3. Also, as mentioned above, in kidney transplant (N=33; CCFZ533X2201-Part 2), after the loading period (non steady state conditions), the mean observed trough plasma CFZ533 concentration on Day 57 was 306 μg/mL (range 161-419 μg/mL; not shown in FIG. 3.

The CFZ5533 300 mg weekly SC regimen for T1DM patients with body weight >50 kg (Category III) has recently been evaluated in Primary Sjögren's Syndrome patients from Study CCFZ533X2203-Cohort 3 (N=25; after an IV loading dose or after a SC loading regimen).

The predicted steady state median CFZ533 trough concentration of 222 μg/mL for T1DM patients/subjects in Category III is:
similar to the mean trough level observed in Primary Sjögren's Syndrome subjects in Study CCFZ533X2203-Cohort 2 (10 mg/kg IV regimen; (i) in FIG. 3), and
slightly above the mean Ctrough value observed for CFZ533 in Study CCFZ533X2203-Cohort 3 ((vi) in FIG. 3). This expected difference is the consequence of an expected slightly higher subcutaneous bioavailability for CFZ533 in T1DM as compared to Primary Sjögren's Syndrome. Indeed, due to elevated CD40 levels in the pre-systemic compartment (likely the lymphatic system), a lower SC bioavailability of CFZ533 is expected in Primary Sjögren's Syndrome subjects as the consequence of a pre-systemic CD40-mediated elimination of CFZ533. In new onset T1DM patients/subjects, high CD40 expression is expected in the inflamed pancreas, and the pre-systemic pool of CD40 receptors likely lower as compared to Primary Sjögren's Syndrome subjects.

Similar steady state trough plasma concentrations of CFZ533 are predicted for Category I (135 mg SC weekly), II (195 mg SC weekly) and III (300 mg SC weekly).

The predicted maximum CFZ533 plasma concentration at steady state (median Cmax,ss) is about 294 μg/mL (body weight Category III; predicted interval: 207 (5th percentile)−453 (95th percentile) g/mL). These Cmax's are at least 19-fold lower than Cmax,ss values measured in the 13-week or 26-week Toxicology studies in non-human primates.

Predicted plasma CFZ533 exposures in T1DM patients/subjects are within observed exposures demonstrated to be efficacious in Primary Sjögren's Syndrome and kidney transplant subjects.
In Primary Sjögren's Syndrome subjects, mean trough plasma concentrations of about 203 μg/mL (Day 113) and 135 μg/mL (Day 141; end of treatment period) in Study CCFZ533X2203-Cohort 2 (10 mg/kg IV regimen), were associated with clinical efficacy at 12 and 24 weeks (clear improvement of the European League Against Rheumatism Sjögren's Syndrome Disease Activity Index (ESSDAI) in CFZ533 treated subjects as compared to placebo). Overall, multiple doses of 10 mg/kg IV CFZ533 in Primary Sjögren's Syndrome subjects, including a total of eight doses over 21 weeks, have been safe and well tolerated.
In Study CCFZ533X2201-Part 2 (10 mg/kg IV loading regimen up to Day 57, then 10 mg/kg IV every 4 weeks up to last dose on Day 337—Mean Ctrough of ca. 156 μg/mL at Day 337) CFZ533 plasma concentrations were well tolerated and efficacious. Subjects in the CFZ533-treated arm had significantly better renal function throughout the study (the difference in eGFR was approximately 10 mL/min) and the risk for acute rejection was similar to that of subjects treated with tacrolimus (standard-of-care arm).

### Example 5. Non-Clinical Pharmacokinetics and Pharmacodynamics

### 1. Pharmacokinetics (PK)

Typical for IgG immunoglobulins, the primary route of elimination of mAb1 is likely via proteolytic catabolism, occurring at sites that are in equilibrium with plasma. In addition, binding and internalization of mAb1-CD40 complexes resulted in rapid and saturable clearance routes. This was illustrated by non-linear mAb1 serum concentration-time profiles showing an inflection point at about 10-20 μg/mL. The contribution of the CD40-mediated clearance to the overall clearance depends on mAb1 concentration, together with levels of CD40 expression, internalization and receptor turnover rates. For serum concentrations of mAb1 ≥10-20 μg/mL, linear kinetics are expected, while non-linear kinetics emerged at lower concentrations.

### 2. Pharmacodynamics (PD)

In a PK/PD study in cynomolgus monkeys, the inflection point (about 10 μg/mL) in the PK profiles was associated with a drop of CD40 saturation, as determined in an independent lymphocyte target saturation assay. As such this inflection point is viewed as a marker for the level of saturation of CD40, and an evidence for target engagement.

The link between CD40 occupancy and pharmacodynamic activity was further demonstrated in rhesus monkeys immunized with KLH. Monkeys were immunized with KLH three times (the first was about 3 weeks prior to dosing, the second was 2 weeks after mAb1 administration, and the third was after complete wash-out of mAb1). CD40 occupancy by mAb1 at plasma concentrations >40 μg/mL at the time of the second KLH vaccination completely prevented recall antibody responses. Once mAb1 was cleared, all animals mounted a full memory antibody response to the third KLH. These results suggest that the function of preexisting memory B cells were not affected. After complete elimination of mAb1, immunization with tetanus toxoid (TTx) led to anti-TTx-IgG/IgM titers similar to non-treated animals and demonstrated that full TDAR was regained after mAb1 elimination.

### 3. Immunogenicity

As expected from an immunosuppressive drug, immunogenicity data in rhesus monkey (single dose) are in agreement with the results from the KLH-TDAR experience and confirmed that no immune response against mAb1 could be mounted under full CD40 occupancy by mAb1.

### Example 6. Characterization of the In Vitro and In Vivo Properties of CFZ533, a Blocking and Non-Depleting Anti-CD40 Monoclonal Antibody

### 1. Methods

Surface Plasmon Resonance Analysis of Affinity of CFZ533 for CD40

The binding analyses of recombinant CFZ533 were performed at 25° C. with HBS-EP+ as running buffer. A typical binding analysis cycle consisted of three steps: (i) capture of the antibody via ProteinA immobilized on the chip surface, (ii) binding of CD40 antigen to the captured anti-CD40 antibody, and (iii) regeneration of the ProteinA surface. To determine the kinetic rate constants of the antigen-antibody binding interactions, binding data were processed, double referenced with responses from blank injections. The binding curves were fitted locally using the 1:1 interaction model of the Biacore T100 Evaluation software to determine kinetic rate constants. The value for the equilibrium dissociation constant (KD) was calculated as the ratio of the rate constants kd/ka. All binding measurements were performed in two independent experiments.

Surface Plasmon Resonance Analysis of Affinity of CFZ533 for FcγRIIA

Extracellular domains of human FcγRIIIA tagged with a 4-amino acid purification tag (4APP; Novartis) and an Avi biotinylation tag (GLNDIFEAQKIEWHE; Avidity) were synthesized by Geneart: human FcγRIIIA (CD16a) 158V (Uniprot: P08637, 17-199), human FcγRIIIA 158F (Uniprot: P08637, 17-199), expressed in HEK293 cells and purified with anti-4APP affinity chromatography. Receptors were site directed biotinylated with BirA (Avidity), bound to streptavidin sensor chips (General Electric), and the equilibrium-binding levels of the different Abs were analyzed by surface plasmon resonance (T100, General Electric) as described (Warncke et al. 2012). Equilibrium dissociation constants (K_{D}) were calculated by a 1:1 model.

Human Leukocyte Cultures

Whole blood buffy coats were obtained from healthy volunteers. Human tonsil samples were obtained from both Ergolz Klinik (Liestal, Switzerland) (Study Protocol No. 1000244 v.03; approved by Ethikkommission beider Basel; EKBB) and Kantonspital (Liestal, Switzerland) (Study Protocol No. TRI0149 v.01; approved by EKNZ). For in vitro culture experiments, please see supplementary material for detailed methods. Briefly, whole blood, isolated PBMCs, in vitro derived monocyte DCs or human tonsil B cells were incubated with single concentrations or a dose titration of CFZ533 or relevant control antibodies. For pathway blocking experiments, these cultures also included an EC80 concentration of recombinant human CD154 (5 μg/ml) and IL-4 (75 ng/ml). Readouts for in vitro assays included proliferation assessed by thymidine incorporation (³H-TdR), flow cytometric-based assessment of expression of the activation molecule CD69 on B cells, and cytokine secretion assessed by ELISA. Similar assays were used for NHP whole blood and PBMCs. In some human whole blood experiments, CD40 receptor occupancy was also examined by used of a fluorescently tagged CFZ533. Where appropriate, IC50 values were estimated using linear regression-based curve-fitting in GraphPad Prism® software.

In Vitro Cell Depletion Assays

See supplementary material for detailed methods. Briefly, the ability of CFZ533 to mediated depletion of CD20^{pos} B cells was monitored in human whole blood over a period of three days in comparison to the B cell depleting antibody Rituximab. For CDC, CFZ533 was incubated with RAJI B cells in the presence or absence of rabbit complement and cell lysis was assessed by luminescence.

Internalization of CFZ533

Internalization of fluorescently tagged CFZ533 and rCD154 was assessed in vitro using the human B cell line RI-1 (Th'ng et al, 1987). CD40 dependence of CFZ533 internalization was assessed using a CD40 knockout RI-1 cell line. Internalization was assessed using an Amnis® image flow cytometer (Merck KHaA, Darnstadt) according to the manufacturer's instructions and data analyzed using ImageStream®X software.

In Vivo Studies

Single dose pharmacokinetic/pharmacodynamic (PK/PD) studies utilized biologics-treatment naive cynomolgus monkeys (Macaca fascicularis) between 7.5-8.5 years old (6.5±2.6 kg) and captive-bred from Philippines (Siconbrec, Makati City, Philippines). The study was performed according to an authorized study protocol and local standard operating procedures in strict compliance with national legal regulations on animal welfare law and accepted animal welfare standards.

In the PK study, CFZ533 was administered to three animals at calculated single doses of 16.2 (5532), 18.5 (5531) and 20 (5530) mg/kg. Blood was sampled for analyses of CFZ533 serum concentrations, numbers of peripheral T and B lymphocytes, and CD40 occupancy on peripheral B cells by CFZ533. For recall TDAR experiments, animals were immunized with keyhole limpet hemocyanin (KLH) in Alum on study days 8 (priming) and 43 (recall; during CFZ533 treatment) respectively. Serum was sampled one day before and 7, 14 and 21 days after priming and recall immunizations. KLH specific IgM/IgG titers were determined with sandwich ELISA using cynomolgus monkey anti-KLH IgM/IgG reference serum as standard. PK assessment was performed as described above. See supplementary material for additional details on the PK and TDAR experiments.

Histological Analysis of Germinal Centers

Sections of formalin fixed, embedded in paraffin wax (FFPE) spleen and lymph nodes (axillary, mandibular and mesenteric) stained with hematoxylin and eosin as well as with an indirect immune-peroxidase method (HRP+DAB from Dako) with the following markers: anti-CD20 antibody (M0755, Dako), anti-CD8 antibody (RM-9116-SO, Medac) and Ki67 (M7240, Dako). All slides were assessed and graded according to the intensity of the staining (negative to intense). In addition, the staining pattern and distribution of any immunohistochemical stained cells within the tissue were also described.

### Example 7. CFZ533 Binds Human CD40 and Inhibits rCD154-Induced Activation of Multiple CD40 Expressing Cell Types

Table 3 indicates that the KD of CFZ533 for recombinant human CD40 was determined by surface plasmon resonance as 0.3 nM, thus being very similar to its parental antibody HCD122 (wild-type IgG1 version of CFZ533).

TABLE 3
Binding affinities (KD) and kinetics
of HCD122 and CFZ533 to human CD40.

| | HCD122 | CFZ533 |
|---|---|---|
| K_{D} [M] | 4.67 ± 1.00 × 10⁻¹⁰ | 3.05 ± 0.26 × 10⁻¹⁰ |
| kₐ [1/Ms] | 2.84 ± 0.67 × 10⁵ | 3.13 ± 0.73 × 10⁵ |
| k_{d} [1/s] | 1.26 ± 0.03 × 10⁻⁴ | 0.93 ± 0.14 × 10⁻⁴ |
| Chi²[RU²] | 0.17-0.19 | 0.10-0.15 |

FIG. 4A shows effect of CFZ533 on rCD154 and IL-4-mediated proliferation (3H-TdR) of human whole blood cultures, PBMCs, and isolated tonsil B cells from multiple donors (5, 32 and 6 donors respectively). Data is presented as normalized cpm (rCD154+IL-4=100; dotted lines). FIG. 4B shows CFZ533 inhibited TNF-alpha production by rCD154-stimulated moDCs after overnight culture. FIG. 4C shows delayed addition of CFZ533 inhibited rCD154+IL-4 mediated human PBMC proliferation. CFZ533 was added to human PBMCs one hour before, simultaneously with, or two and six hours after stimulation with rCD154+IL-4, and proliferation (3H-TdR) was assessed after a subsequent four days of culture (dotted and dashed lines represent rCD154+IL-4 and cell plus media controls). For all data, the mean and SD of readouts of rCD154-induced stimulation were graphed as a function of log-transformed CFZ533 concentrations. Where appropriate, IC50 values were determined using linear regressionbased curve-fitting. FIG. 4D shows relationship between CD40 occupancy and pathway blockade by CFZ533. Human whole blood from 10 donors was cultured overnight with rCD154 in presence of a dose titration of CFZ533. The degree of pathway activation (% CD69pos on B cells) and degree of CD40 occupancy (staining with AlexaFlour 488 labeled CFZ533) was evaluated. Open and filled circles indicate the percent of CD40 occupied by CFZ533 and percent CD69pos expressing cells on CD20pos B cells as a function of log-transformed CFZ533 concentration respectively (Mean and SD shown). Dotted and dashed lines represent rCD154-induced CD69 expression and cells plus media control cultures normalized across all donors.

FIG. 4 indicates that CFZ533 completely inhibited rCD154-induced proliferation of human whole blood cultures, PBMCs as well as purified tonsillar B cells from multiple donors with potencies (IC50 values) of 0.024 μg/ml (0.16 nM), 0.017 μg/ml (0.12 nM) and 0.071 μg/ml (0.47 nM) respectively. In addition, we could demonstrate that CFZ533 completely blocked rCD154-induced TNF production by primary monocyte-derived dendritic cells (moDCs) with an IC50 of 0.04 μg/ml (0.27 nM) (FIG. 4B).

As published previously, CFZ533 inhibited rCD154-induced proliferation of PBMCs from Cynomolgus monkeys (Cordoba et al., 2015). CFZ533 inhibited rCD154-induced proliferation of PBMCs from humans, rhesus and cynomolgus animals with similar potency (IC50 of 0.02, 0.03, and 0.01 μg/ml, respectively), and could also bind CD40 on B cells from these species with EC50 values of approximately 0.2 μg/ml, see Table 4.

TABLE 4
Cellular binding and functional properties
of CFZ533 in human and NHPs.

| | Inhibition of rCD154- | | CD40 occupancy by | |
|---|---|---|---|---|
| | induced proliferation (IC50 | | CFZ533 (MFI EC50 on | |
| | PBMCs) | | CD20+ cells) | |
| Human | 0.017 + 0.012 | μg/ml | 0.22 + 0.042 | μg/ml |
| | 0.12 + 0.08 | μM | 1.49 + 0.28 | μM |
| | (n = 32) | | (n = 4) | |
| Rhesus | 0.026 + 0.017 | μg/ml | 0.22 + 0.033 | μg/ml |
| | 0.18 + 0.12 | μM | 1.49 + 0.22 | μM |
| | (n = 8) | | (n = 6) | |
| Cynomolgus | 0.010 + 0.003 | μg/ml | 0.20 + 0.068 | μg/ml |
| | 0.07 + 0.02 | μM | 1.35 + 0.46 | μM |

| | |
|---|---|
| (n = 4) | (n = 4) |

The above cellular data were derived from experiments where CFZ533 was added prior to, or simultaneously with rCD154, indicating that the antibody could prevent binding of the endogenous ligand. We could also demonstrate that addition of CFZ533 up to 6 hours following initiation of leukocyte cultures containing rCD154 resulted in complete inhibition of cellular activation with minimal loss of potency, indicating that CFZ533 could displace the endogenous ligand from CD40 (FIG. 4C).

We also wanted to evaluate the relationship between the degree of CD40 occupancy by CFZ533, and the extent of pathway inhibition. To do so we simultaneously assessed CD40 receptor occupancy by CFZ533 and rCD154-induced CD69 in whole blood from multiple donors. FIG. 4D indicates that CD40 receptor occupancy by CFZ533 of at least 90% was required for complete blockade of CD40 pathway activation. A similar relationship between receptor occupancy and pathway inhibition was also observed using CD23 and CD54 as readouts of CD40 pathway activation (data not shown).

### Example 8: CFZ533 Displays Minimal Stimulatory Potential In Vitro

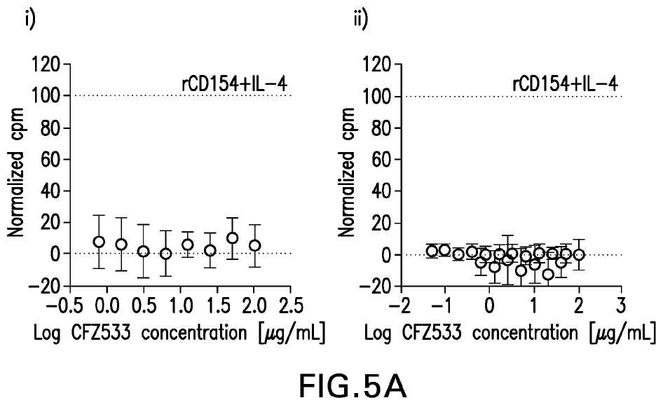

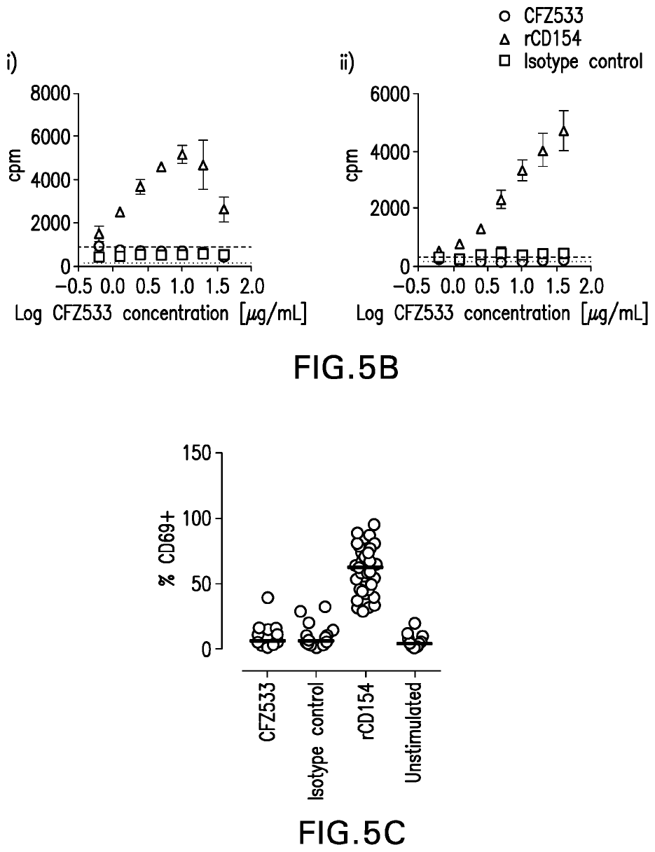

The ability of CFZ533 to stimulate activation of human leukocytes was assessed using proliferation and upregulation of the activation molecule CD69 on B cells in whole blood. FIG. 5A shows data regaring i. Human whole blood from multiple donors (n=13) were incubated with a dose titration of CFZ533, and proliferation (³H-TdR) was assessed after three days of culture. ii. Human PBMCs from multiple donors (n=26) were incubated with a dose titration of CFZ533, and proliferation (³H-TdR) was assessed after three days of culture. For both graphs, data is presented as mean and SD of normalized cpm as a function of log-transformed CFZ533 concentration (rCD154+IL-4=100; dotted lines, cells plus media=0; dashed lines). FIG. 5B shows that CFZ533 does not induce human PBMC proliferation in the presence of additional stimuli. Human PBMCs were stimulated for 3 days with a dose titration of CFZ533 in the presence of IL-4 (i) or anti-IgM F(ab′)2. (ii). The mean and SD of 3H-TdR (cpm) is shown as a function of log-transformed CFZ533 concentration. In FIG. 5C it is shown how human whole blood (41 donors) was cultured overnight with no stimuli, CFZ533, isotype control or rCD154 and CD69 expression on B cells was assessed by FACS. Each dot represents data from a single donor with mean % CD69 values indicated by a horizontal red line.

FIG. 5A shows that CFZ533 was unable to induce thymidine incorporation by human whole blood (1:10 dilution) or PBMCs in contrast to rCD154. The inability of CFZ533 to induce proliferation was unaffected by the addition of additional co-stimuli such as IL-4, or anti-IgM (FIG. 5B). We could also demonstrate that CFZ533 was unable to induce upregulation of CD69 on B cells in whole blood from multiple donors, again in contrast to rCD154 (FIG. 5C). Finally, CFZ533 was unable to induce cytokine production by CD40 expressing monocyte-derived DCs or human umbilical vein endothelial cells (HUVECs) (data not shown).

### Example 9: CFZ533 does not Mediate Cell Depletion

CFZ533 was engineered to contain a N297A mutation, previously demonstrated to abrogate FcγR binding resulting in an inability to mediate antibody-dependent cellular cytotoxicity (ADCC). CFZ533 was not able to bind FcγRIIIA in comparison to HCD122 (wild-type IgG1) (Table 5), and we wanted to examine how this lack of binding affected the ability of CFZ533 to mediate cell depletion.

TABLE 5
Binding affinities (kₐ[1/M]) of HCD122
and CFZ533 to human FcγRIIIA

| | HCD122 | CFZ533 |
|---|---|---|
| FcγR species | (wild-type IgG1) | (N297A IgG1) |
| Human FcγRIIIA 158V | 1.72 × 106 | n.d. |
| Human FcγRIIIA 158F | 6.99 × 105 | n.d. |

n.d. not detected

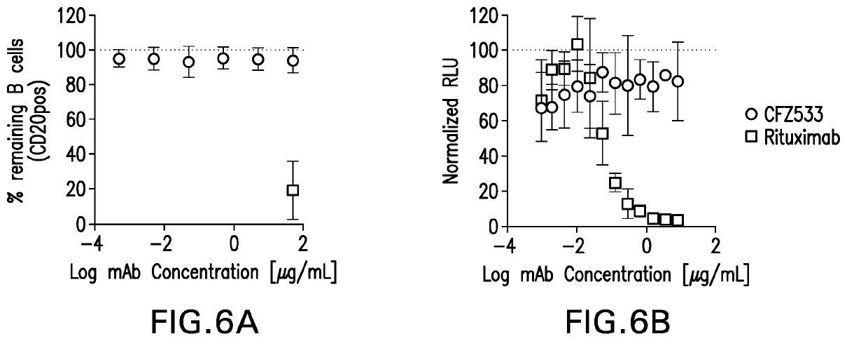

FIG. 6A shows data from human whole blood cultures incubated for 72 hours in the presence of a dose titration of CFZ533 or 50 μg/ml Rituximab. B cells numbers were determined based on CD45pos and CD19pos events falling within lymphocyte FSC/SSC gate. Results for individual antibody concentrations were calculated as percent remaining B cells with reference to untreated samples and graphed as a function of log-transformed antibody concentration (adjusted to 100% and shown as a dotted line). Data represent the mean and SD of eight independent donors. FIG. 6B shows results from Raji B cells incubated with different concentrations of Rituximab or CFZ533 and a fixed concentration of rabbit complement. Concentration dependent killing of the Raji cells was analyzed after 2 hours, where the viability of the cells was measured by determination of the ATP concentration in each well using luciferase. Results are presented as isotype-control normalized relative luciferase units (RLU) as a function of log-transformed antibody concentration.

FIG. 6A indicates that while the depleting anti-CD20 antibody Rituximab was able to eliminate approximately 80% of B cells in human whole blood, while CFZ533 failed to mediate any cell depletion. In addition, CFZ533 was unable to mediate complement-dependent cytotoxicity (CDC) of Raji B cells, in contrast to Rituximab (FIG. 6B).

### Example 10: CFZ533 is Internalized by B Cells in a CD40-Dependent Manner

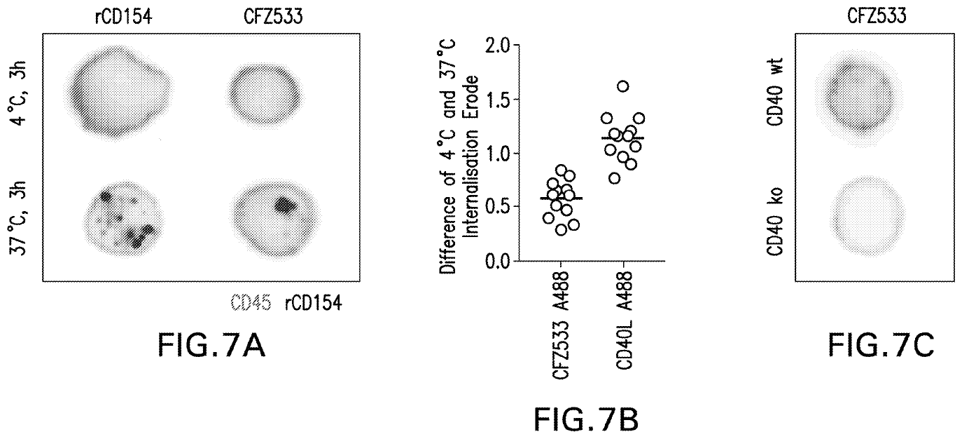

We next wanted to examine whether CFZ533 could be internalized by the CD40 expressing human B cell line RI-1. FIG. 7A indicates that rCD154 was internalized under permissive conditions (37° C.) in comparison to non-permissive conditions (4° C.), where weak staining of rCD154 could be observed on the plasma membrane. CFZ533 was also internalized, although there did appear to be residual membrane staining at 37° C. FIG. 7B indicated that the extent of internalization of rCD154 appeared to be greater than that observed for CFZ533. Using a CD40 knockout RI-1 B cell line, we could demonstrate that binding and internalization of CFZ533 (FIG. 7C) and rCD154 (data not shown) was CD40 dependent.

FIG. 7A shows Representative images of individual RI-1 B cells cultured with AlexaFlour 488 labeled rCD154 or CFZ533 for 3 hours at 37° C. or 4° C. FIG. 7B. Relative internalization erode of CFZ533 and rCD154 under permissive conditions (non-permissive erode values subtracted). Each dot represents data from an individual experiment and the population mean is indicated as a horizontal red line. FIG. 7C. Representative images of individual CD40 expressing or CD40 knock-out RI-1 cells cultured with Alexa488 labeled CFZ533 for 3 hours at 37° C. In all experiments, cells were co-stained with AlexaFlour 647 labeled CD45 to demark the cell membrane.

### Example 11: Pharmacokinetic Properties of CFZ533 in Non-Human Primates

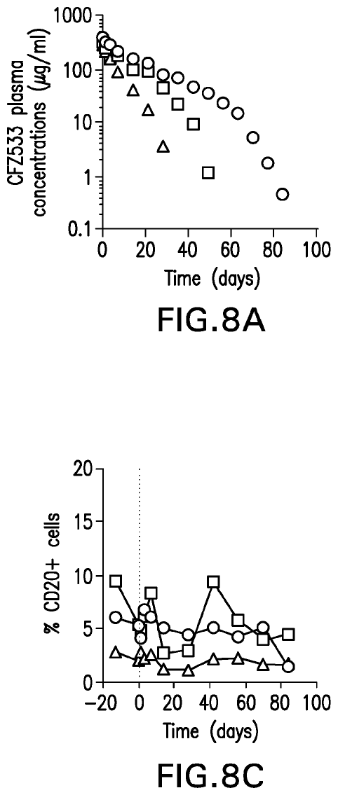

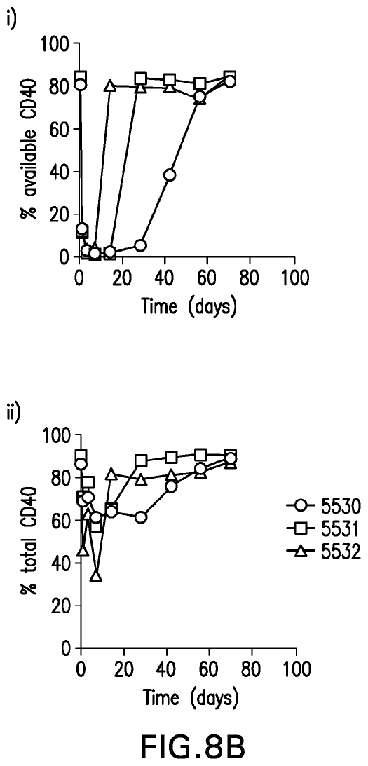

FIG. 8A. Serum concentrations of CFZ533 in three cynomolgus monkeys after single dose administration at calculated doses of 16.2 (5532), 18.5 (5531) and 20 (5530) mg/kg intravenously. FIG. 8B. CD40 occupancy: percent available CD40 (i) and percent total CD40 (ii) C. Peripheral B/T cells: percentage of peripheral blood B cells after single dose. Day 0 is when CFZ533 was administered.

Data above indicated that CFZ533 bound NHP CD40, and could inhibit rCD154-induced activation of NHP B cells with similar potencies. This suggested that cynomolgus and rhesus monkeys would be suitable species for in vivo studies investigating the relationship between CFZ533 PK and PD. Data in FIG. 8A shows the PK profiles of three cynomolgus monkeys following a single intravenous dose of CFZ533 (calculated doses of 16.2, 18.5 and 20 mg/kg). Typical for a monoclonal antibody targeting an internalizing membrane bound antigen (Mager et al. 2006 and Ng et al. 2006), the time course of CFZ533 concentration exhibited clear target-mediated disposition, resulting in non-linear PK profiles and concentration-dependent clearance rate and half-life. The inflection point observed in the PK profiles is a marker of target engagement and is associated with an increased contribution of CD40 to the overall clearance of CFZ533, and a shorter half-life. Further, the inflection point in the PK profiles coincided with the time where a drop of CD40 saturation was observed (FIG. 8B, i). This occurred at approximately 10-20 μg/ml, when CFZ533 was subject to more rapid elimination. In all animals, there was no loss of CD40 receptor expression on cells (FIG. 8B, ii). Further, CFZ533 did not deplete peripheral blood B cells (FIG. 8C) or T cells (data not shown), despite some observed variations throughout the study.

### Example 12: CFZ533 Inhibits Recall T Cell-Dependent Antibody Production

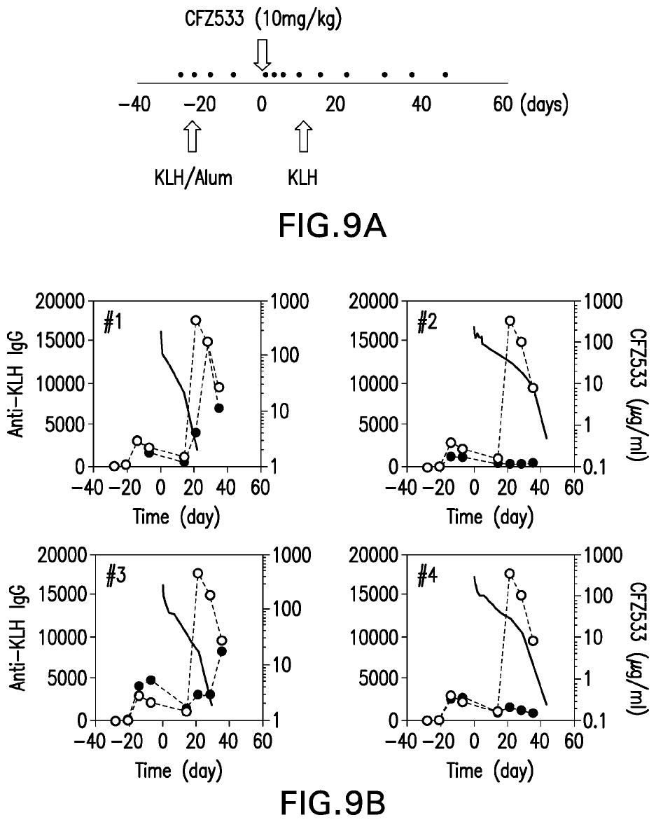

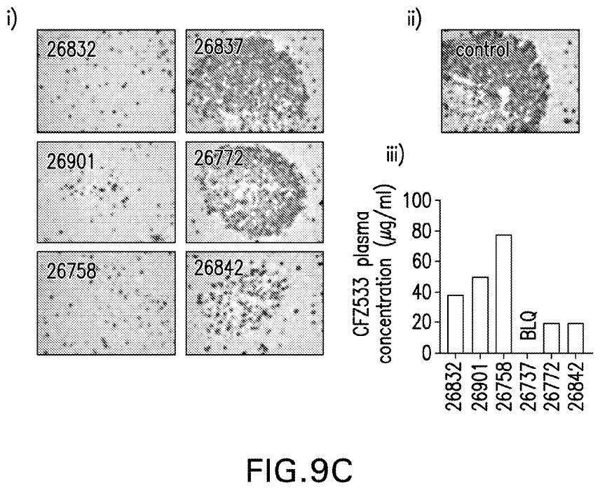

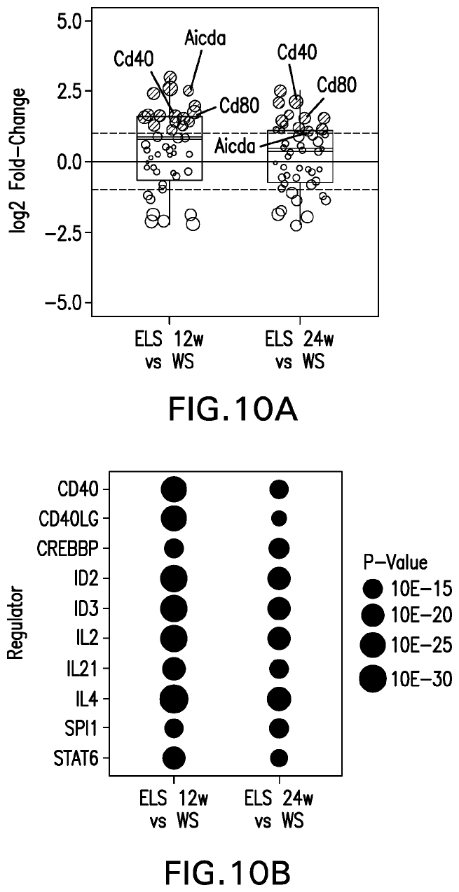

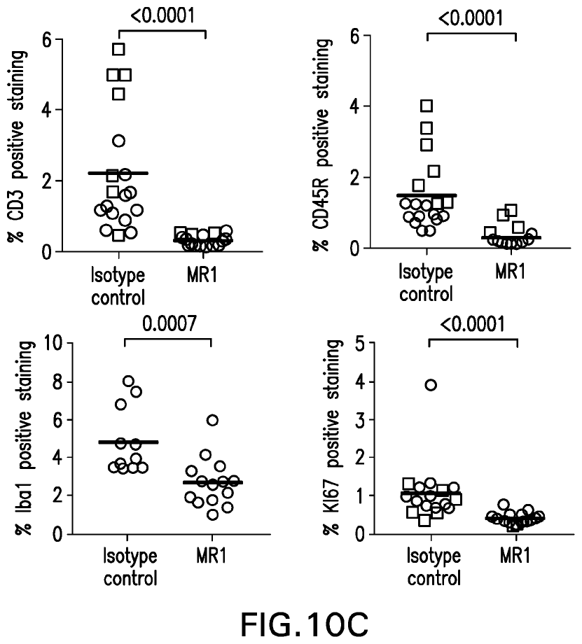

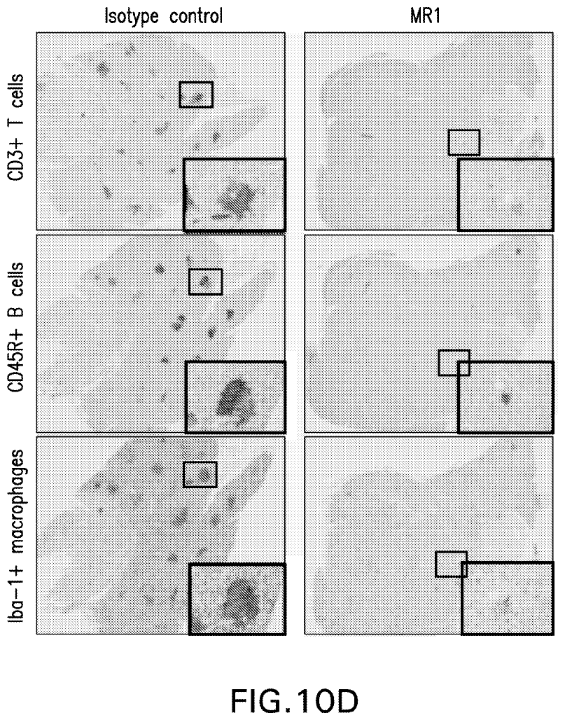

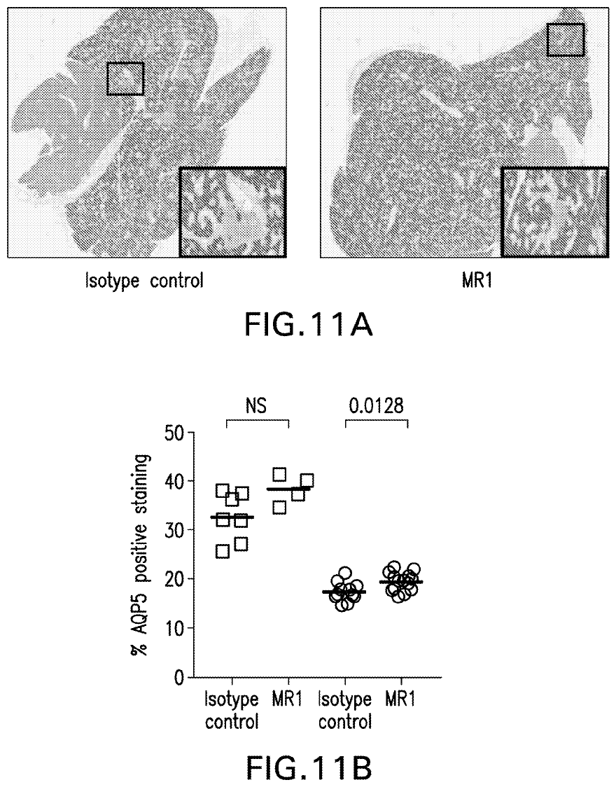

FIG. 9A shows experimental design schematic for evaluating the effect of CFZ533 on recall TDARs. Arrows below the x-axis highlight primary and secondary KLH immunizations. The timing of a single dose of 10 mg/kg CFZ533 is shown above. The asterisks indicate time points at which anti-KLH IgG and/or CFZ533 levels were measured. FIG. 9B. Each graph shows anti-KLH IgG (closed symbols) and plasma CFZ533 levels (log-scale; unbroken line) for an individual animal. Average anti-KLH IgG levels from control animals (open symbols) are overlaid on each graph for comparative purposes. FIG. 9C. Histological analysis of germinal centers (Ki67 staining) in mLNs from Rhesus monkeys from a 1 mg/kg/week subcutaneous multiple dose 26-week study using CFZ533. Representative mLN sections from six animals are shown (i) along with a control image (ii). iii. Average steady state CFZ533 serum concentrations over a dosing interval from individual animals at the end of the treatment period.

An expected on-target, PD effect of CD40 blocked is inhibition of a TDAR (Kawabe et al. 1994). CFZ533 inhibits primary TDARs in NHPs and humans, and we also wanted to examine the effects of this antibody on a recall TDAR. The experimental design is summarized in FIG. 9A. Briefly, four rhesus monkeys were immunized with KLH in Alum at study day −28 (priming), prior to a single intravenous dose of CFZ533 at 10 mg/kg on study day 1, followed by a second KLH immunization on study day 15.

FIG. 9B illustrates the effects of CFZ533 on anti-KLH IgG recall responses in four individual animals in comparison to data from immunized controls (no CFZ533). There was inter-animal variability in PK profiles of CFZ533, with more rapid elimination of CFZ533 observed in animals #1 and #3. Higher plasma concentrations were observed for a longer period of time in animals #2 and #4. Interestingly, these animals displayed complete suppression of an anti-KLH IgG (and IgM; data not shown) recall response on study day (note all animals mounted a primary TDAR to KLH). In contrast, anti-KLH IgG responses were observed (albeit with some delay) in animals with more rapid clearance of CFZ533 (higher delay for animal #3 as compared to animal #1), notably when serum CFZ533 levels were less than approximately 40 μg/ml at the time of second KLH immunization. As has been observed with previous in vivo experiments with CFZ533 in transplanted (Cordoba et al. 2015) and nontransplanted animals (FIG. 8B), no peripheral B cell depletion was observed (data not shown).

The above results indicated that CFZ533 serum concentrations higher than approximatively 40 μg/ml were required for complete suppression of a recall TDAR in NHPs. We wanted to further examine the relationship between CFZ533 exposure and CD40 pathway-relevant tissue pharmacodynamic effects. At the termination of a 26-week toxicology study, at 1 mg/kg/week CFZ533 subcutaneously we performed histological and molecular analysis of GCs in mesenteric lymph nodes (mLNs). FIG. 9C (i) indicates that of the six animals dosed, we could observe complete suppression of GCs in three individuals, whereas GCs could still be observed in the mLNs of the remaining animals. FIG. 9C (iii) indicates that serum concentrations of at least 38 μg/mL (average steady-state concentration over the dosing interval) were associated with complete suppression of GC development in cortical B cell areas of lymph nodes, whereas incomplete (animal 26842) or no suppression (animals 26772 and 26837) of GCs was observed at serum concentrations below 20 μg/mL, despite full CD40 occupancy on whole blood CD20^{pos} B cells (animals 26842 and 26772; data not shown). There was no evidence of peripheral B cell depletion (data not shown).

### ADDITIONAL PREFERRED EMBODIMENTS

A. An anti-CD40 antibody with silenced ADCC activity for use in the treatment of insulitis, comprising administering a therapeutically effective amount of said antibody to a patient in need thereof, wherein said antibody is administered through a loading dosing followed by a maintenance dosing and the route of administration is subcutaneous or intravenous, or a combination of subcutaneous or intravenous.

B. An antibody for use according to embodiment A, wherein the loading dosing is administered via intravenous injection as a first dose and the maintenance dosing is administered via subcutaneous injections as a second dose being different from the first dose.

C. An antibody for use according to embodiments A or B, wherein the loading dose is about 3 mg to about 60 mg antibody per kilogram of the patient.

D. An antibody for use according to embodiment A to C, wherein the patient is a pediatric patient.

E. An antibody for use according to embodiment D, wherein the loading dose is 30 mg/kg administered intravenously at day 1 and the maintenance dose is administered subcutaneously as a fixed dose between 100 mg to 350 mg once weekly starting at day 8.

F. An antibody for use according to embodiment E, wherein the maintenance dose is administered subcutaneously as a fixed dose by body weight weekly starting at day 8 at a dose of:

a. 135 mg for patients of body weight category I, having a bodyweight between 20 kg to 30 kg;
b. 195 mg for patients of body weight category II, having a bodyweight between 30 kg to 50 kg; and
c. 300 mg for patients of body weight category III, having a bodyweight above 50 kg.

G. An antibody for use according to embodiment F, wherein
a. the patients of body weight category I will receive each maintenance dose in form of a single injection of 0.9 ml; and
b. the patients of body weight category II will receive each maintenance dose in form of a single injection of 1.3 ml; or
c. the patients of body weight category III will receive each maintenance dose in form of a single injection of 2 ml or 2 injections of 1 ml.

H. An antibody for use according to embodiments A to G, wherein the treatment is continued up to 52 weeks after day 1.

I. An antibody for use according to embodiment D, wherein the age range of the patients is between 6 and 21 years.

J. The antibody for use according to any of the previous embodiments A to I, wherein the antibody is selected from the group consisting of:

a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14. (35)

K. The antibody for use according to embodiment J, wherein the antibody comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

L. The antibody for use according to embodiment J or K, wherein the antibody is CFZ533.

M. A pharmaceutical composition comprising a therapeutically effective amount of the antibody for use according to any of embodiment A to L and one or more pharmaceutically acceptable carriers.

N. A method of treating insulitis in a human subject, comprising administering a therapeutically effective dose of an anti-CD40 antibody with silenced ADCC activity to said subject, wherein said antibody is administered through a loading dosing followed by a maintenance dosing and the route of administration is subcutaneous or intravenous, or a combination of subcutaneous or intravenous.

O. The method according to embodiment N, wherein the loading dosing is administered via intravenous injections of a first dose and the maintenance dosing is administered via subcutaneous injections of a second dose being different from the first dose.

P. The method according to embodiment 0, wherein the loading dose is about 3 mg to about 60 mg antibody per kilogram of the patient.

Q. The method according to embodiments N-O, wherein the patient is a pediatric patient.

R. The method according to embodiment Q, wherein the loading dose is 30 mg/kg administered intravenously at day 1 and the maintenance dose is administered subcutaneously as a fixed dose between 100 mg-350 mg once weekly starting at day 8.

S. The method according to embodiment R, wherein the maintenance dose is administered subcutaneously as a fixed dose by body weight weekly starting at day 8 at a dose of:

a. 135 mg for patients of body weight category I, having a bodyweight between 20 to 30 kg;
b. 195 mg for patients of body weight category II, having a bodyweight between 30 to 50 kg; and
c. 300 mg for patients of body weight category III, having a bodyweight above 50 kg.

T. The method according to embodiment 5, wherein

a. the patients of body weight category I will receive each maintenance dose in form of a single injection of 0.9 ml; and
b. the patients of body weight category II will receive each maintenance dose in form of a single injection of 1.3 ml; or
c. the patients of body weight category III will receive each maintenance dose in form of a single injection of 2 ml or 2 injections of 1 ml.

U. The method according to embodiment T, wherein the treatment is continued for up to 52 weeks after day 1.

V. The method according to embodiments Q-U, wherein the age range of the patients is between 6 and 21 years.

W. The method according to embodiment N-V, wherein the antibody is selected from the group consisting of:

a. an anti-CD40 antibody comprising an immunoglobulin VH domain comprising the amino acid sequence of SEQ ID NO: 7 and an immunoglobulin VL domain comprising the amino acid sequence of SEQ ID NO: 8; b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

X. The method of treatment according to embodiment W, wherein the antibody comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

Y. The method of treatment according to embodiments W or X, wherein the antibody is CFZ533.

Z. Use of a liquid pharmaceutical composition comprising an anti-CD40 antibody with silenced ADCC activity, a buffer, a stabilizer and a solubilizer, for the manufacture of a medicament for the treatment of T1DM, wherein the anti-CD40 antibody is selected from the group consisting of:

1. an anti-CD40 antibody comprising an immunoglobulin VH domain comprising the amino acid sequence of SEQ ID NO: 7 and an immunoglobulin VL domain comprising the amino acid sequence of SEQ ID NO: 8;

2. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;

3. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;

4. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14; and

5. an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

AA. Use of a liquid pharmaceutical composition comprising an anti-CD40 antibody with silenced ADCC activity, a buffer, a stabilizer and a solubilizer, for the manufacture of a medicament for the treatment of insulitis, wherein the anti-CD40 antibody is selected from the group consisting of:

1. an anti-CD40 antibody comprising an immunoglobulin VH domain comprising the amino acid sequence of SEQ ID NO: 7 and an immunoglobulin VL domain comprising the amino acid sequence of SEQ ID NO: 8;

2. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;

3. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13;

4. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14; and

5. an anti-CD40 antibody comprising the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

### REFERENCES

Andersen ML, Hougaard P, Porksen S, et al. (2014) Partial Remission Definition: Validation based on the insulin dose-adjusted HbA1c (IDAA1C) in 129 Danish Children with New-Onset Type 1 Diabetes. Pediatr Diabetes (15): 469-476.
Andrassy J, Zeier M, Andrassy K (2004). Do we need screening for thrombophilia prior to kidney transplantation? Nephrol Dial Transplant: 19(Suppl 4): iv64.
Astorri E, Bombardieri M, Gabba S, et al (2010). Evolution of ectopic lymphoid neogenesis and in situ autoantibody production in autoimmune nonobese diabetic mice: cellular and molecular characterization of tertiary lymphoid structures in pancreatic islets. J Immunol; 185(6):3359-68.
Balasa B, Krahl T, Patstone G, et al (1997). CD40 ligand-CD40 interactions are necessary for the initiation of insulitis and diabetes in nonobese diabetic mice. J Immunol; 159(9):4620-7.
Borrok M. J., Mody N., Lu X., Kuhn M. L., Wu H. An “Fc silenced” IgG1 format with extended half-life designed for improved stability. J. of Pharmaceutical Sciences 106, 1008-10017)
Bundy B N, Krischer J P (2016). A model-based approach to sample size estimation in recent onset type 1 diabetes. Diabetes Metab Res Rev; 32(8):827-834.
Bottazzo G F, Pujol-Borrell R, Hanafusa T, et al (1983) Role of aberrant HLA-DR expression and antigen presentation in induction of endocrine autoimmunity. Lancet. 1115-9.
Cengiz E, Cheng P, Ruedy K J, et al (2017). Clinical outcomes in youth beyond the first year of type 1 diabetes: Results of the Pediatric Diabetes Consortium (PDC) type 1 diabetes new onset (NeOn) study. Pediatr Diabetes. 18(7):566-573.
Chatzigeorgiou A, Harokopos V, Mylona-Karagianni C, et al (2010a) The pattern of inflammatory/anti-inflammatory cytokines and chemokines in type 1 diabetic patients over time. Ann. Med. 426-38.
Chatzigeorgiou A E, Lembessis P E, Mylona-Karagianni C F, et al (2010b) CD40 expression and its association with low-grade inflammation in a Greek population of type 1 diabetic juveniles: evidence for differences in CD40 mRNA isoforms expressed by peripheral blood mononuclear cells. Exp. Clin. Endocrinol. Diabetes. 38-46.
Cho N H, Shaw J E, Karuranga S, et al (2018) IDF Diabetes Atlas: Global estimates of diabetes prevalence for 2017 and projections for 2045. Diabetes Res. Clin. Pract. 271-281.
Christie M R. (2016) Delving Into the Type 1 Diabetic Islet: Evidence That B-Cell Infiltration of Islets Is Linked to Local Hyperimmunity and Accelerated Progression to Disease. Diabetes; 65.5: 1146-1148.
Couper J, Donaghue K C (2009) Phases of diabetes in children and adolescents. Pediatr Diabetes. 13-6.
Devaraj S, Glaser N, Griffen S, et al (2006). Increased monocytic activity and biomarkers of inflammation in patients with type 1 diabetes. Diabetes. 55(3):774-9.
Diabetes Control and Complications Trial Research Group (1994). Effect of intensive diabetes treatment on the development and progression of long-term complications in adolescents with insulin-dependent diabetes mellitus: Diabetes Control and Complications Trial. J Pediat; 125:177-188.
Eshima K, Mora C, Wong F S, et al (2003). A crucial role of CD4 T cells as a functional source of CD154 in the initiation of insulin-dependent diabetes mellitus in the non-obese diabetic mouse. Int Immunol.15(3):351-7.
Goldwater R, Keirns J, Blahunka P, et al (2013). A phase 1, randomized ascending single-dose study of antagonist anti-human CD40 ASKP1240 in healthy subjects. Am J Transplant. 13(4):1040-1046.
Greenbaum C J, Beam C A, Boulware D, et al (2012) Fall in C-peptide during first 2 years from diagnosis: evidence of at least two distinct phases from composite Type 1 Diabetes TrialNet data. Diabetes. 2066-73.
Haase C, Markholst H (2007). CD40 is required for development of islet inflammation in the RIP-CD154 transgenic mouse model of type 1 diabetes. Ann N Y Acad Sci. 1107:373-9.
Hao W, Gitelman S, DiMeglio L A, et al (2016) Fall in C-Peptide During First 4 Years From Diagnosis of Type 1 Diabetes: Variable Relation to Age, HbA1c, and Insulin Dose. Diabetes Care. 1664-70.
Harland R., Klintmalm G., Jensik S., et al (2017). Efficacy and Safety of Bleselumab in Kidney Transplant Recipients: A Phase 2, Randomized, Open-Label Study. Am J Transplant. 17.
Huck K, Feyen O, Ghosh S, et al (2009). Memory B-cells in healthy and antibody-deficient children. Clinical Immunology: 131(1):50-59.
Iezzi G, Sonderegger I, Ampenberger F, et al (2009) CD40-CD40L cross-talk integrates strong antigenic signals and microbial stimuli to induce development of IL-17-producing CD4+ T cells. Proc. Natl. Acad. Sci. U.S.A. 876-81.
Imperatore G, Boyle J P, Thompson T J, et al (2012) Projections of type 1 and type 2 diabetes burden in the U.S. population aged <20 years through 2050: dynamic modeling of incidence, mortality, and population growth. Diabetes Care. 2515-20.
Johnston D (2004) NICE guidance on type 1 diabetes in adults. Clin Med (Lond). 491-3.
Klein D, Barbé-Tuana F, Pugliese A, et al (2005). A functional CD40 receptor is expressed in pancreatic beta cells. Diabetologia. 48(2):268-76.
Katsarou A, Gudbjörnsdottir S, Rawshani A, et al (2017). Type 1 diabetes mellitus. Nat Rev Dis Primers.3:17016.
Kawai T, Andrews D, Colvin R B, Sachs D H, Benedict Cosimi A. (2000).
Thromboembolic complication after treatement with monoclonal antibody against CD40 ligand. Nature Medicine, Vol. 6, No. 2
Leete P, Willcox A, Krogvold L, et al (2016).
Differential Insulitic Profiles Determine the Extent of 3-Cell Destruction and the Age at Onset of Type 1 Diabetes. Diabetes. 65(5):1362-9.
Leighton E, Sainsbury C A, Jones G C (2017) A Practical Review of C-Peptide Testing in Diabetes. Diabetes Ther. 475-487.
Ma A, Dun H, Song L, et al (2014). Pharmacokinetics and pharmacodynamics of ASKP1240, a fully human anti-CD40 antibody, in normal and renal transplanted Cynomolgus monkeys. Transplantation.97(4):397-404.
Miller K M, Foster N C, Beck R W, et al (2015) Current state of type 1 diabetes treatment in the U.S.: updated data from the T1DM Exchange clinic registry. Diabetes Care. 971-8.
Mortensen H B, Hougaard P, Swift P, et al (2009) New definition for the partial remission period in children and adolescents with type 1 diabetes. Diabetes Care. 1384-90.
Pescovitz M D, Greenbaum C J, Bundy B, et al (2014). B-lymphocyte depletion with rituximab and j-cell function: two-year results. Diabetes Care. 2014 February; 37(2):453-9.
Peters N C, Kroeger D R, Mickelwright S, et al (2009). CD4 T cell cooperation is required for the in vivo activation of CD4 T cells. Int Immunol.21(11):1213-24.
Price J D, Beauchamp N M, Rahir G, et al (2014). CD8+ dendritic cell-mediated tolerance of autoreactive CD4+ T cells is deficient in NOD mice and can be corrected by blocking CD40L. J Leukoc Biol. 95(2):325-36.
Secrest A M, Becker D J, Kelsey S F, et al. (2010) Cause-Specific Mortality Trends in a Large Population-Based Cohort With Long-Standing Childhood-Onset Type 1 Diabetes. Diabetes; 59(12): 3216-3222.
Shearer W T, Rosenblatt H M, Gelman R S, et al (2003) Lymphocyte subsets in healthy children from birth through 18 years of age: the Pediatric AIDS Clinical Trials Group P1009 study. J. Allergy Clin. Immunol. 973-80.
Shock A, Burkly L, Wakefield I, et al (2015). CDP7657, an anti-CD40L antibody lacking an Fc domain, inhibits CD40L-dependent immune responses without thrombotic complications: an in vivo study. Arthritis Res Ther. 17:234.
Simon A K, Hollander G A, McMichael A (2015). Evolution of the immune system in humans from infancy to old age. Proc Biol Sci.; December 22; 282(1821): 20143085.
Steffes M W, Sibley S, Jackson M, et al (2003). Beta-cell function and the development of diabetes-related complications in the diabetes control and complications trial. Diabetes Care. 26(3):832-6.
Swinnen S G, Mullins P, Miller M, et al (2009). Changing the glucose cut-off values that define hypoglycaemia has a major effect on reported frequencies of hypoglycaemia. Diabetologia 52: 38-41
Thomas L. Chapter 18.10 Immunoglobulins (2012). In: Clinical laboratory diagnostics: use and assessment of clinical laboratory results/Lothar Thomas (editor), 667-678. ISBN 978-3-9805215-4-3.
Vaitaitis G M, Olmstead M H, Waid D M, et al (2014). A CD40-targeted peptide controls and reverses type 1 diabetes in NOD mice. Diabetologia. 57(11):2366-73.
Vaitaitis G M, Waid D M, Yussman M G, et al (2017). CD40-mediated signalling influences trafficking, T-cell receptor expression, and T-cell pathogenesis, in the NOD model of type 1 diabetes. Immunology. 152(2):243-254.
Viisanen T, Ihantola E L, Nanto-Salonen K, et al (2017). Circulating CXCR5+PD-1+ICOS+ Follicular T Helper Cells Are Increased Close to the Diagnosis of Type 1 Diabetes in Children With Multiple Autoantibodies. Diabetes. 66(2):437-447.
Wagner D H (2017). Overlooked Mechanisms in Type 1 Diabetes Etiology: How Unique Costimulatory Molecules Contribute to Diabetogenesis. Frontiers in Endocrinology; 8:208:1 10.
Wang D D, Zhang S, Zhao H, et al (2009) Fixed dosing versus body size-based dosing of monoclonal antibodies in adult clinical trials. J Clin Pharmacol. 1012-24.
Wang W, Prueksaritanont T (2010) Prediction of human clearance of therapeutic proteins: simple allometric scaling method revisited. Biopharm Drug Dispos. 253-63.
Wang Q, Racine J J, Ratiu J J, et al (2017). Transient BAFF Blockade Inhibits Type 1 Diabetes Development in Nonobese Diabetic Mice by Enriching Immunoregulatory B Lymphocytes Sensitive to Deletion by Anti-CD20 Cotherapy. J Immunol.199(11):3757-3770.
Wherrett D K, Chiang J L, Delamater A M, et al (2015) Defining pathways for development of disease-modifying therapies in children with type 1 diabetes: a consensus report. Diabetes Care. 1975-85.
Woittiez N J, Roep B O (2015) Impact of disease heterogeneity on treatment efficacy of immunotherapy in Type 1 diabetes: different shades of gray. Immunotherapy. 163-74.
Xu X, Shi Y, Cai Y, et al (2013) Inhibition of increased circulating Tfh cell by anti-CD20 monoclonal antibody in patients with type 1 diabetes. PLoS ONE. e79858. Workgroup on Hypoglycemia, American Diabetes Association (2005). Defining and reporting hypoglycemia in diabetes: a report from the American Diabetes Association Workgroup on Hypoglycemia. Diabetes Care, 28(5):1245-9.

1.-13. (canceled)

14. A method of treating type 1 diabetes mellitus (T1DM) in a human subject, comprising administering a therapeutically effective dose of an anti-CD40 antibody with silenced antibody dependent cell-mediated cytotoxic (ADCC) activity to said subject, wherein said antibody is administered through a loading dosing followed by a maintenance dosing and the route of administration is subcutaneous or intravenous, or a combination of subcutaneous or intravenous.

15. The method according to claim 14, wherein the loading dosing is administered via intravenous injections of a first dose and the maintenance dosing is administered via subcutaneous injections of a second dose being different from the first dose.

16. The method according to claim 15, wherein the loading dose is about 3 mg to about 60 mg antibody per kilogram of the patient.

17. The method according to claim 14, wherein the patient is a pediatric patient.

18. The method according to claim 14, wherein the loading dose is 30 mg/kg administered intravenously at day 1 and the maintenance dose is administered subcutaneously as a fixed dose between 100 mg-350 mg once weekly starting at day 8.

19. The method according to claim 18, wherein the maintenance dose is administered subcutaneously as a fixed dose by body weight once weekly starting at day 8 at a dose of:
a. 135 mg for patients of body weight category I, having a bodyweight between 20 to 30 kg;
b. 195 mg for patients of body weight category II, having a bodyweight between to 50 kg; and
c. 300 mg for patients of body weight category III, having a bodyweight above 50 kg.

20. The method according to claim 19, wherein
a. the patients of body weight category I will receive each maintenance dose in form of a single injection of 0.9 ml; and
b. the patients of body weight category II will receive each maintenance dose in form of a single injection of 1.3 ml; or
c. the patients of body weight category III will receive each maintenance dose in form of a single injection of 2 ml or 2 injections of 1 ml.

21. The method according to claim 20, wherein the treatment is continued for up to 52 weeks after day 1.

22. The method according to claim 17, wherein the age range of the patients is between 6 and 21 years.

23. The method according to claim 14, wherein the antibody is selected from the group consisting of:
a. an anti-CD40 antibody comprising an immunoglobulin VH domain comprising the amino acid sequence of SEQ ID NO: 7 and an immunoglobulin VL domain comprising the amino acid sequence of SEQ ID NO: 8;
b. an anti-CD40 antibody comprising an immunoglobulin VH domain comprising the hypervariable regions set forth as SEQ ID NO: 1, SEQ ID NO: 2, and SEQ ID NO: 3 and an immunoglobulin VL domain comprising the hypervariable regions set forth as SEQ ID NO: 4, SEQ ID NO: 5 and SEQ ID NO: 6;
c. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 13; and
d. an anti-CD40 antibody comprising an immunoglobulin VH domain comprising the amino acid sequence of SEQ ID NO: 7 and an immunoglobulin VL domain comprising the amino acid sequence of SEQ ID NO: 8, and an Fc region of SEQ ID NO: 14.

24. The method of treatment according to claim 23, wherein the antibody comprises the heavy chain amino acid sequence of SEQ ID NO: 9 and the light chain amino acid sequence of SEQ ID NO: 10; or the heavy chain amino acid sequence of SEQ ID NO: 11 and the light chain amino acid sequence of SEQ ID NO: 12.

25. The method of treatment according to claim 23, wherein the antibody is CFZ533.

26. (canceled)

27. The method of treatment according to claim 14, wherein the patient is treated within 100 days after diagnosis of T1 DM.


SEQUENCE LISTING (19 sequences)

SEQ ID NO: 1 - protein, 5 aa, Homo sapiens
SYGMH

SEQ ID NO: 2 - protein, 17 aa, Homo sapiens
VISYEESNRYHADSVKG

SEQ ID NO: 3 - protein, 11 aa, Homo Sapiens
DGGIAAPGPDY

SEQ ID NO: 4 - protein, 16 aa, Homo sapiens
RSSQSLLYSNGYNYLD

SEQ ID NO: 5 - protein, 7 aa, Homo sapiens
LGSNRAS

SEQ ID NO: 6 - protein, 9 aa, Homo sapiens
MQARQTPFT

SEQ ID NO: 7 - protein, 120 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 8 - protein, 112 aa, Homo sapeins
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 9 - protein, 450 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 219 aa, Homo sapiens
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 450 aa, Homo sapiens
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 219 aa, Homo sapiens; identical to SEQ ID NO: 10

SEQ ID NO: 13 - protein, 217 aa, Homo sapiens
APELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 217 aa, Homo sapiens
APELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1350 bp, Homo sapiens
caggtgcagctggtggaatctggcggcggagtggtgcagcctggccggtccctgagactgtcttgcgccgcctccggcttcaccttctccagctacggcatgcactgggtgcgacaggcccctggcaagggactggaatgggtggccgtgatctcctacgaggaatccaacagataccacgctgactccgtgaagggccggttcacaatctcccgggacaactccaagatcaccctgtacctgcagatgaactccctgcggaccgaggacaccgccgtgtactactgcgccagggacggaggaatcgccgctcctggacctgattattggggccagggcaccctggtgacagtgtcctccgctagcaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgaccgtgccctctagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacgcctccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccacaggtgtacacactgccccccagccgggaagagatgaccaagaaccaggtgtccctgacctgtctggtcaaaggcttctacccctccgatatcgccgtggagtgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 16 - DNA, 657 bp, Homo sapiens
gacatcgtgatgacccagtcccccctgtccctgaccgtgacacctggcgagcctgcctctatctcctgcagatcctcccagtccctgctgtactccaacggctacaactacctggactggtatctgcagaagcccggccagtccccacaggtgctgatctccctgggctccaacagagcctctggcgtgcccgaccggttctccggctctggctctggcaccgacttcacactgaagatctcacgggtggaagccgaggacgtgggcgtgtactactgcatgcaggcccggcagacccccttcaccttcggccctggcaccaaggtggacatccggcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 17 - DNA, 1350 bp, Homo sapiens
caggtgcagctggtggaatctggcggcggagtggtgcagcctggccggtccctgagactgtcttgcgccgcctccggcttcaccttctccagctacggcatgcactgggtgcgacaggcccctggcaagggactggaatgggtggccgtgatctcctacgaggaatccaacagataccacgctgactccgtgaagggccggttcacaatctcccgggacaactccaagatcaccctgtacctgcagatgaactccctgcggaccgaggacaccgccgtgtactactgcgccagggacggaggaatcgccgctcctggacctgattattggggccagggcaccctggtgacagtgtcctccgctagcaccaagggcccctccgtgttccctctggccccctccagcaagtccacctctggcggcaccgccgctctgggctgcctggtgaaagactacttccccgagcccgtgaccgtgtcctggaactctggcgccctgacctccggcgtgcacacctttccagccgtgctgcagtcctccggcctgtactccctgtcctccgtggtgaccgtgccctctagctctctgggcacccagacctacatctgcaacgtgaaccacaagccctccaacaccaaggtggacaagcgggtggaacccaagtcctgcgacaagacccacacctgtcccccctgccctgcccctgaactgctgggcggaccttccgtgttcctgttccccccaaagcccaaggacaccctgatgatctcccggacccccgaagtgacctgcgtggtggtggccgtgtcccacgaggaccctgaagtgaagttcaattggtacgtggacggcgtggaagtgcacaacgccaagaccaagcccagagaggaacagtacaactccacctaccgggtggtgtctgtgctgaccgtgctgcaccaggactggctgaacggcaaagagtacaagtgcaaggtctccaacaaggccctgcctgcccccatcgaaaagaccatctccaaggccaagggccagccccgcgagccacaggtgtacacactgccccccagccgggaagagatgaccaagaaccaggtgtccctgacctgtctggtcaaaggcttctacccctccgatatcgccgtggagtgggagtccaacggacagcccgagaacaactacaagaccaccccccctgtgctggactccgacggctcattcttcctgtactccaagctgaccgtggacaagtcccggtggcagcagggcaacgtgttctcctgctccgtgatgcacgaggccctgcacaaccactacacccagaagtccctgtccctgagccccggcaag

SEQ ID NO: 18 - DNA, 657 bp, Homo sapiens; identical to SEQ ID NO: 16

SEQ ID NO: 19 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ